(19) United States
(12) Patent Application Publication
(10) Pub. No.: US 2024/0358866 A1
(43) Pub. Date: Oct. 31, 2024

(54) NSAID-DERIVED CONJUGATE COMPOUNDS FOR IMAGING

(71) Applicant: Reiley Pharmaceuticals, Inc., San Francisco, CA (US)

(72) Inventors: B. Michael SILBER, Mill Valley, CA (US); Frank KAYSER, San Rafael, CA (US); Mark A. REILEY, Delray Beach, FL (US)

(21) Appl. No.: 18/523,771

(22) Filed: Nov. 29, 2023

Related U.S. Application Data
(60) Provisional application No. 63/429,051, filed on Nov. 30, 2022.

(51) Int. Cl. A61K 51/04; G01N 33/60

(52) U.S. Cl. CPC A61K 51/0497; A61K 51/0446; A61K 51/0453; A61K 51/0485; G01N 33/60; G01N 2800/102; G01N 2800/2842; G01N 2800/52

(57) ABSTRACT

Compounds derived from celecoxib, valdecoxib, and indomethacin, and methods of use thereof, are disclosed. The compounds are useful for, inter alia, identifying and localizing the site of pathology and/or inflammation responsible for the sensation of pain in a patient; for identifying the sites of primary, secondary, benign, or malignant tumors; and for diagnosing infection or confirming or ruling out suspected infection. The compounds contain a radioactive agent which permits imaging. The compounds concentrate at sites of increased cyclooxygenase expression, such as areas of increased COX-2 expression, thus revealing the sites of increased prostaglandin production, which is correlated with pain and inflammation, and correlated with tumor presence and/or location. Identifying areas of increased COX expressing can also aid in screening for infections, assessing efficacy of diagnosis and treatment of rheumatoid arthritis, and assessing the need for treatment with opioid drugs.

### CROSS-REFERENCE TO RELATED APPLICATIONS

This application claims priority to U.S. Provisional Patent Application No. 63/429,051, filed Nov. 30, 2022, the contents of which are incorporated by reference herein in their entirety.

### FIELD OF THE INVENTION

Conjugate compounds derived from valdecoxib, celecoxib, and indomethacin, and methods of use thereof, are disclosed. Such compounds are useful for, inter alia, identifying and localizing the site of pathology and/or inflammation responsible for the sensation of pain in a patient, sites of infection, and identifying and localizing the sites of tumor pathology, including benign, malignant, primary, and secondary tumors.

### BACKGROUND OF THE INVENTION

It is important in medicine to identify the site of pathology in order to properly diagnose, screen for, and/or treat a disease. Tumor screening for the presence of tumors (e.g. for breast cancer, cervical cancer, colon cancer, prostate cancer, etc.) is very common. Some of the difficulties with tumor screening are expense, patient's time, physician's time, and accuracy. Also, many of the screening tests are not particularly accurate. For example, testing for prostate cancer using serum acid phosphatase or prostate specific antigen (PSA) is non-specific, and elevation of the marker in healthy individuals can be cause for an unnecessary surgery, a prostate biopsy. An additional example is MRI screening for breast tumors, whose value has recently been questioned for both insensitivity and occasional misinterpretation. In addition, the presence or absence of sentinel (metastatic) nodes is critical for the optimal treatment of breast cancer. Low grade chondrosarcomas are notoriously difficult to read by the pathologist, and frequently have to be sent to multiple institutions for a diagnostic consensus. All of these examples suggest the need for improving detection for all benign, malignant, primary and secondary tumors. A rapid and non-invasive method of localizing tumors would aid immensely in diagnosing and treating the underlying cause. The growing tendency to understand tumors at the molecular level may also be guided by such improved non-invasive methods.

Localization of pain is another area where identifying the site of pathology is important for treatment; however, such localization is often not straightforward. The unpleasant sensation of pain serves as an indicator of a disease or pathological state. Pain often occurs at the site of pathology and can be a helpful guide in determining diagnosis and appropriate treatment. However, in many cases, the area where a patient experiences pain may not be coincident with the area where the actual pathology has occurred. A classic example is sciatica, where pressure on the sciatic nerve due to a herniated disc in the lower spine can result in a sensation of pain in the leg, at a significant distance from the site of pathology. Another example is the difficulty in diagnosing pain in the chest or thorax, which can arise from multiple causes, such as cardiac ischemia, gastroesophageal reflux, or pulmonary embolism, and diagnosis of abdominal pain, which can arise from appendicitis, ischemic bowel disease, abdominal abscess, diverticulitis, Crohn's disease, ulcerative colitis, volvulus, inter alia. In such cases, differential diagnosis requires a systematic process of elimination through tests and procedures until the cause and/or location of pathology is identified.

Screening for infectious diseases, particularly when a patient is still asymptomatic, also poses difficulties. Medicaments and methods for such screening would prove useful in limiting outbreaks of diseases; early treatment of infected individuals; and avoiding unnecessary treatment or isolation for individuals who are suspected of being infected, but who in actuality have not been infected, by a disease.

Because pathology is often accompanied by inflammation at the site of the pathology (which is not necessarily the site where pain is experienced), rapid and non-invasive methods of localizing inflammation in a patient experiencing pain would aid immensely in diagnosing and treating the underlying cause of the pain.

### SUMMARY OF THE INVENTION

The current disclosure provides compounds and methods useful for identification of areas of pathology, including tumors and inflammation, and screening for infections and sites of infections, via non-invasive imaging. All of the compounds and methods disclosed herein can be used in both human and veterinary medicine.

In one embodiment, disclosed herein are conjugate compounds of Formula (I), Formula (II), or Formula (III):

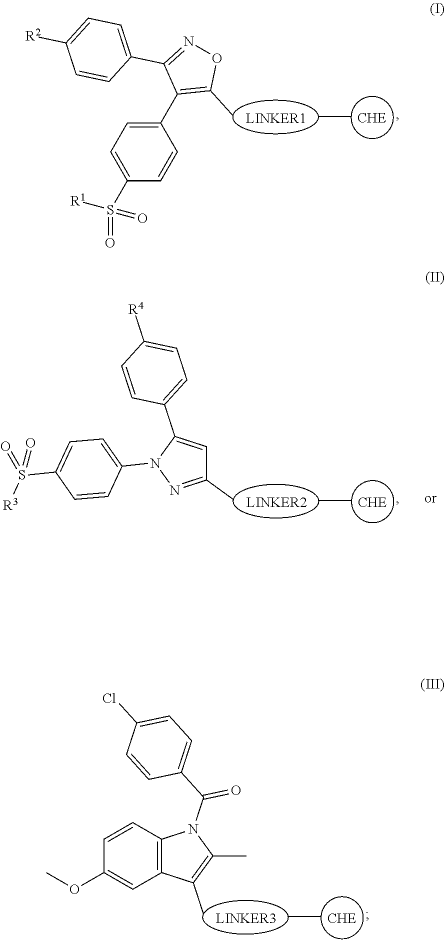

or a salt thereof, wherein

R¹ is —NH₂ or —CH₃;
R² is H, F, Cl, —OCH₃, —CH₃, or —CF₃;
R³ is —NH₂ or —CH₃;
R⁴ is H, F, Cl, —CH₃, —OCH₃, or —CF₃;

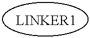

### is —R⁵—;

R⁵ is alkylene, haloalkylene, alkenylene, heteroalkylene, or heteroalkylene substituted with halogen;

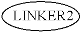

### is —R⁶—;

R⁶ is alkylene, haloalkylene, alkenylene, heteroalkylene, or heteroalkylene substituted with halogen;

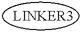

### is —R⁷—;

R⁷ is alkylene, haloalkylene, alkenylene, heteroalkylene, heteroalkylene substituted with halogen, —CH₂—C(═O)NH-alkylene, —CH₂—C(═O)NH-haloalkylene, —CH₂—C(═O)NH-alkenylene, —CH₂—C(═O)NH-heteroalkylene, —CH₂—C(═O)NH-heteroalkylene substituted with halogen, —CH₂—NHC(═O)-alkylene, —CH₂—NHC(═O)-haloalkylene, —CH₂—NHC(═O)-alkenylene, —CH₂—NHC(═O)-heteroalkylene, or —CH₂—NHC(═O)-heteroalkylene substituted with halogen;

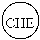

is

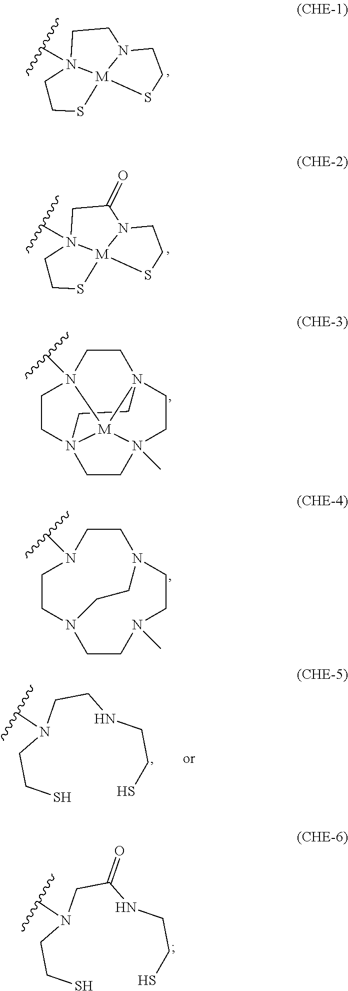

and
M is copper, gallium, zinc, or manganese.

In one embodiment, the compounds are of Formula (I),

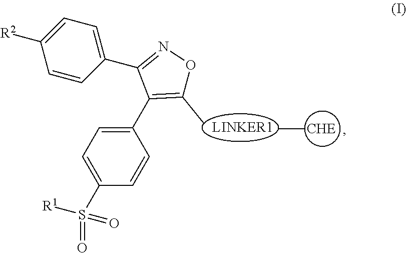

or a salt thereof.

In one embodiment, the compounds are of Formula (II),

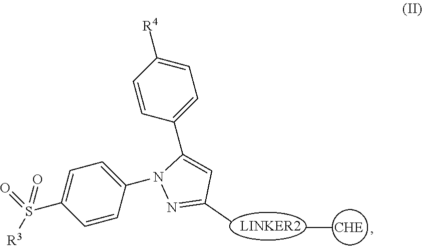

or a salt thereof.

In one embodiment, the compounds are of Formula (III),

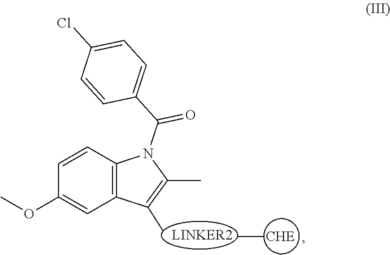

or a salt thereof.

In any embodiments of the compounds disclosed herein, or a salt thereof, M can be copper (Cu), such as Cu(II). In any embodiments of the compounds disclosed herein, or a salt thereof, M can be ⁶⁴Cu. In any embodiments of the compounds disclosed herein, or a salt thereof, M can be ⁶³Cu. In any embodiments of the compounds disclosed herein, or a salt thereof, M can be ⁶⁵Cu. In any embodiments of the compounds disclosed herein, or a salt thereof, M can be copper at natural isotopic abundance (approximately 69.15% ⁶³Cu and 30.85% ⁶⁵Cu). In any embodiments of the compounds disclosed herein, or a salt thereof, M can be ⁶³Cu and ⁶⁵Cu in any ratio. In any embodiments of the compounds disclosed herein, or a salt thereof, M can be gallium (Ga), such as Ga(III). In any embodiments of the compounds disclosed herein, or a salt thereof, M can be ⁶⁸Ga. In any embodiments of the compounds disclosed herein, or a salt thereof, M can be ⁶⁹Ga. In any embodiments of the compounds disclosed herein, or a salt thereof, M can be ⁷¹Ga. In any embodiments of the compounds disclosed herein, or a salt thereof, M can be gallium at natural isotopic abundance (approximately 60.1% ⁶⁹Ga and 39.9% ⁷¹Ga). In any embodiments of the compounds disclosed herein, or a salt thereof, M can be ⁶⁹Ga and ⁷¹Ga in any ratio. In any embodiments of the compounds disclosed herein, or a salt thereof, M can be zinc, such as Zn(II). In any embodiments of the compounds disclosed herein, or a salt thereof, M can be ⁶³Zn. In any embodiments of the compounds disclosed herein, or a salt thereof, M can be ⁶⁴Zn. In any embodiments of the compounds disclosed herein, or a salt thereof, M can be ⁶⁶Zn. In any embodiments of the compounds disclosed herein, or a salt thereof, M can be ⁶⁷Zn. In any embodiments of the compounds disclosed herein, or a salt thereof, M can be ⁶⁸Zn. In any embodiments of the compounds disclosed herein, or a salt thereof, M can be ⁷⁰Zn. In any embodiments of the compounds disclosed herein, or a salt thereof, M can be zinc at natural isotopic abundance (approximately ⁶⁴Zn (48.6%), ⁶⁶Zn (27.9%), ⁶⁷Zn (4.1%), ⁶⁸Zn (18.8%), and ⁷⁰Zn (0.6%)). In any embodiments of the compounds disclosed herein, or a salt thereof, M can be ⁶⁴Zn, ⁶⁶Zn, ⁶⁷Zn, ⁶⁸Zn, and ⁷⁰Zn in any ratio. In any embodiments of the compounds disclosed herein, or a salt thereof, M can be manganese, such as Mn(II). In any embodiments of the compounds disclosed herein, or a salt thereof, M can be ⁵²Mn. In any embodiments of the compounds disclosed herein, or a salt thereof, M can be ^{52m}Mn. In any embodiments of the compounds disclosed herein, or a salt thereof, M can be ^{52g}Mn. In any embodiments of the compounds disclosed herein, or a salt thereof, M can be ⁵⁵Mn.

Any of the embodiments of the compounds disclosed herein, or a salt thereof, can additionally have the limitation of a proviso that the longest chain in —R⁵—, —R⁶—, or —R⁷— has at least four atoms and at most twelve atoms.

In some embodiments of Formula (I), R¹ is —NH₂. In some embodiments of Formula (I), R¹ is —CH₃. In some embodiments of Formula (I), R² is H. In some embodiments of Formula (I), R² is F. In some embodiments of Formula (I), R² is Cl. In some embodiments of Formula (I), R² is —CH₃. In some embodiments of Formula (I), R² is —OCH₃. In some embodiments of Formula (I), R² is —CF₃.

In some embodiments of Formula (II), R³ is —NH₂. In some embodiments of Formula (II), R³ is —CH₃. In some embodiments of Formula (II), R⁴ is H. In some embodiments of Formula (II), R⁴ is F. In some embodiments of Formula (II), R⁴ is Cl. In some embodiments of Formula (II), R⁴ is —CH₃. In some embodiments of Formula (II), R⁴ is —OCH₃. In some embodiments of Formula (II), R⁴ is —CF₃.

In any embodiments of the compounds disclosed herein, or a salt thereof, R⁵, R⁶, or R⁷ can be C₁-C₁₂ alkylene, C₁-C₁₂ haloalkylene, C₂-C₁₂ alkenylene, heteroalkylene having between 2 and 10 carbon atoms and between 1 and 4 heteroatoms selected from O, S, and N (where N in the heteroalkylene chain can be substituted with H or C₁-C₄ alkyl), or heteroalkylene having between 2 and 10 carbon atoms and between 1 and 4 heteroatoms selected from O, S, and N (where N in the heteroalkylene chain can be substituted with H or C₁-C₄ alkyl) substituted with halogen, for example 1, 2, 3, or 4 halogen atoms. In any embodiments of the compounds disclosed herein, or a salt thereof, where R⁵, R⁶, or R⁷ is heteroalkylene, all of the heteroatoms can be O. In any embodiments of the compounds disclosed herein, or a salt thereof, where R⁵, R⁶, or R⁷ is heteroalkylene substituted with halogen, for example 1, 2, 3, or 4 halogen atoms, or perhalogenated, all of the halogen substituents can be fluorine atoms. In any embodiments of the compounds disclosed herein, or a salt thereof, where R⁵, R⁶, or R⁷ is heteroalkylene substituted with halogen, for example 1, 2, 3, or 4 halogen atoms, all of the heteroatoms can be O and all of the halogen substituents can be fluorine atoms.

In any embodiments of the compounds disclosed herein, or a salt thereof, R⁵, R⁶, or R⁷ can be C₄-C₁₀ alkylene, C₄-C₁₀ haloalkylene, C₄-C₁₀ alkenylene, or heteroalkylene having between 2 and 8 carbon atoms and between 1 and 4 heteroatoms selected from O, S, and N (where N in the heteroalkylene chain can be substituted with H or C₁-C₄ alkyl), or heteroalkylene having between 2 and 10 carbon atoms and between 1 and 4 heteroatoms selected from O, S, and N (where N in the heteroalkylene chain can be substituted with H or C₁-C₄ alkyl) substituted with halogen, for example 1, 2, 3, or 4 halogen atoms. In any embodiments of the compounds disclosed herein, or a salt thereof, where R⁵, R⁶, or R⁷ is heteroalkylene, all of the heteroatoms can be O. In any embodiments of the compounds disclosed herein, or a salt thereof, where R⁵, R⁶, or R⁷ is heteroalkylene substituted with halogen, for example 1, 2, 3, or 4 halogen atoms, or perhalogenated, all of the halogen substituents can be fluorine atoms. In any embodiments of the compounds disclosed herein, or a salt thereof, where R⁵, R⁶, or R⁷ is heteroalkylene substituted with halogen, for example 1, 2, 3, or 4 halogen atoms, all of the heteroatoms can be O and all of the halogen substituents can be fluorine atoms.

In any embodiments of the compounds disclosed herein, or a salt thereof, R⁵, R⁶, or R⁷ can be —(CH₂)ₚ₁—, where p1 can be 1, 2, 3, 4, 5, 6, 7, 8, 9, 10, 11, or 12. In any embodiments of the compounds disclosed herein, or a salt thereof, R⁵, R⁶, or R⁷ can be —(CH₂)ₚ₁—, where p1 can be 4, 5, 6, 7, 8, 9, or 10.

In any embodiments of the compounds disclosed herein, or a salt thereof, R⁵, R⁶, or R⁷ can be —[(CH₂)ₚ₂—O]_{q}—(CH₂)ₚ₃—, wherein each p2 and each p3 can be independently 1, 2, 3, or 4; and q can be 1, 2, or 3.

In any embodiments of the compounds disclosed herein, or a salt thereof,

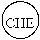

can be

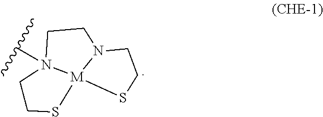

In any embodiments of the compounds disclosed herein, or a salt thereof,

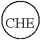

can be

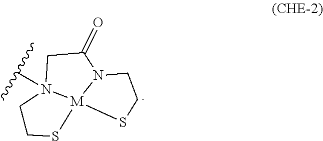

In any embodiments of the compounds disclosed herein, or a salt thereof,

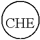

can be

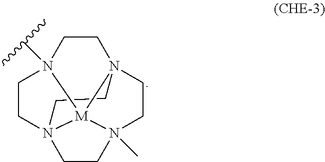

In any embodiments of the compounds disclosed herein, or a salt thereof,

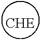

can be

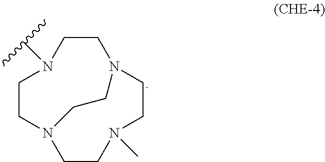 [SMILES: *N1CCN2CCN(C)CCN(CC1)CC2]

In any embodiments of the compounds disclosed herein, or a salt thereof,

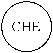

can be

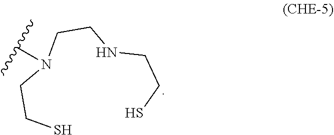 [SMILES: *N(CCS)CCNCCS]

In any embodiments of the compounds disclosed herein, or a salt thereof,

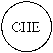

can be

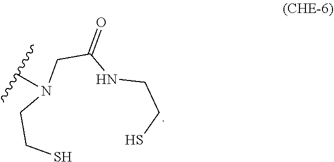 [SMILES: *N(CCS)CC(=O)NCCS]

In any embodiments of the compounds disclosed herein, or a salt thereof, the linker —R⁵—, —R⁶—, or —R⁷— can be:

—(CH₂)₄—,
—(CH₂)₅—,
—(CH₂)₆—,
—(CH₂)₇—,
—(CH₂)₈—,
—(CH₂)₉—,
—(CH₂)₁₀—,
—(CH₂)—O—(CH₂)₄—,
—(CH₂)—O—(CH₂)₅—,
—(CH₂)—O—(CH₂)₆—,
—(CH₂)—O—(CH₂)₇—,
—(CH₂)—O—(CH₂)₃—O—(CH₂)₃—,
—(CH₂)—O—(CH₂)₄—O—(CH₂)₂—,
—(CH₂)—O—(CH₂)—, or
—(CF₂)—(CH₂)₅—.

### Additionally, —R⁷— can be.

—CH₂—C(═O)NH—(CH₂)₄—,
—CH₂—C(═O)NH—(CH₂)₅—,
—CH₂—C(═O)NH—(CH₂)₆—,
—CH₂—C(═O)NH—(CH₂)₇—,
—CH₂—C(═O)NH—(CH₂)₈—,
—CH₂—C(═O)NH—(CH₂)₉—,
—CH₂—C(═O)NH—(CH₂)₁₀—,
—CH₂—C(═O)NH—(CH₂)—O—(CH₂)₄—,
—CH₂—C(═O)NH—(CH₂)—O—(CH₂)₅—,
—CH₂—C(═O)NH—(CH₂)—O—(CH₂)₆—,
—CH₂—C(═O)NH—(CH₂)—O—(CH₂)₇—,
—CH₂—C(═O)NH—(CH₂)—O—(CH₂)₃—O—(CH₂)₃—,
—CH₂—C(═O)NH—(CH₂)—O—(CH₂)₄—O—(CH₂)₂—,
—CH₂—C(═O)NH—(CH₂)—O—(CH₂)₇—,
—CH₂—C(═O)NH—(CF₂)—(CH₂)₅—,
—CH₂—NH—C(═O)—(CH₂)₄—,
—CH₂—NH—C(═O)—(CH₂)₅—,
—CH₂—NH—C(═O)—(CH₂)₆—,
—CH₂—NH—C(═O)—(CH₂)₇—,
—CH₂—NH—C(═O)—(CH₂)₈—,
—CH₂—NH—C(═O)—(CH₂)₉—,
—CH₂—NH—C(═O)—(CH₂)₁₀—,
—CH₂—NH—C(═O)—(CH₂)—O—(CH₂)₄—,
—CH₂—NH—C(═O)—(CH₂)—O—(CH₂)₅—,
—CH₂—NH—C(═O)—(CH₂)—O—(CH₂)₆—,
—CH₂—NH—C(═O)—(CH₂)—O—(CH₂)₇—,
—CH₂—NH—C(═O)—(CH₂)—O—(CH₂)₃—O—(CH₂)₃—,
—CH₂—NH—C(═O)—(CH₂)—O—(CH₂)₄—O—(CH₂)₂—,
—CH₂—NH—C(═O)—(CH₂)—O—(CH₂)₇—, or
—CH₂—NH—C(═O)—(CF₂)—(CH₂)₅—.

In some embodiments, the conjugate compound, or a salt thereof, can have an IC₅₀ for cyclooxygenase inhibition of less than about 0.5 micromolar. The cyclooxygenase can be COX-2.

In further embodiments, disclosed herein is a pharmaceutical composition comprising one or more compounds of any of the conjugate compounds disclosed herein, or a salt thereof, and a pharmaceutically acceptable excipient.

In further embodiments, disclosed herein is a method of imaging a site of pathology or suspected pathology in a subject, comprising: a) administering one or more conjugate compounds disclosed herein, or a salt thereof, or a pharmaceutical composition of any of the foregoing, to the subject, wherein M is ⁶⁴Cu; and b) generating an image of the subject or an image of a portion of the subject. The pathology or suspected pathology in the subject can be a tumor or a suspected tumor. The subject can be suffering from pain. The pathology or suspected pathology in the subject can be an infection or a suspected infection.

In further embodiments, disclosed herein is a method of imaging a site of pathology or suspected pathology in a subject, comprising: a) administering one or more conjugate compounds disclosed herein, or a salt thereof, or a pharmaceutical composition of any of the foregoing, to the subject, wherein M is ⁶⁸Ga; and b) generating an image of the subject or an image of a portion of the subject. The pathology or suspected pathology in the subject can be a tumor or a suspected tumor. The subject can be suffering from pain. The pathology or suspected pathology in the subject can be an infection or a suspected infection.

In further embodiments, disclosed herein is a method of imaging a site of pathology or suspected pathology in a subject, comprising: a) administering one or more conjugate compounds disclosed herein, or a salt thereof, or a pharmaceutical composition of any of the foregoing, to the subject, wherein M is ⁶³Zn; and b) generating an image of the subject or an image of a portion of the subject. The pathology or suspected pathology in the subject can be a tumor or a suspected tumor. The subject can be suffering from pain. The pathology or suspected pathology in the subject can be an infection or a suspected infection.

In further embodiments, disclosed herein is a method of imaging a site of pathology or suspected pathology in a subject, comprising: a) administering one or more conjugate compounds disclosed herein, or a salt thereof, or a pharmaceutical composition of any of the foregoing, to the subject, wherein M is ⁵²Mn (for example, ^{52m}Mn, ^{52g}Mn, or a mixture of ^{52m}Mn and ^{52g}Mn; and b) generating an image of the subject or an image of a portion of the subject. The pathology or suspected pathology in the subject can be a tumor or a suspected tumor. The subject can be suffering from pain. The pathology or suspected pathology in the subject can be an infection or a suspected infection.

In further embodiments, disclosed herein is one or more conjugate compounds, or a salt thereof, or a pharmaceutical composition of any of the foregoing, for use in imaging a site of pathology or suspected pathology in a subject. The pathology or suspected pathology in the subject can be a tumor or a suspected tumor. The subject can be suffering from pain. The pathology or suspected pathology in the subject can be an infection or a suspected infection.

In further embodiments, disclosed herein is the use of one or more conjugate compounds disclosed herein, or a salt thereof, or a pharmaceutical composition of any of the forgoing, in the preparation of a medicament for use in imaging a site of pathology or suspected pathology in a subject. The pathology or suspected pathology in the subject can be a tumor or a suspected tumor. The subject can be suffering from pain. The pathology or suspected pathology in the subject can be an infection or a suspected infection. In further embodiments, the present disclosure provides any of the NSAID derivative compounds disclosed herein, with the substitution of a non-radioactive agent for the radioactive agent. Thus, for any of the generic structures or specific compounds disclosed herein containing ⁶⁴Cu, or its oxides or carbonyl derivatives, the present disclosure also embraces those generic structures or specific compounds with a non-radioactive copper isotope, such as ⁶³Cu or ⁶⁵Cu, or with copper at natural isotopic abundance (approximately 69.15% ⁶³Cu and 30.85% ⁶⁵Cu), or with ⁶³Cu and ⁶⁵Cu in any ratio, or their oxides or carbonyl derivatives. For any of the generic structures or specific compounds disclosed herein containing ⁶⁸Ga, the present disclosure also embraces those generic structures or specific compounds with a non-radioactive gallium isotope, such as ⁶⁹Ga or ⁷¹Ga, or with gallium at natural isotopic abundance (approximately 60.1% ⁶⁹Ga and 39.9% ⁷¹Ga), or with ⁶⁹Ga and ⁷¹Ga in any ratio. For any of the generic structures or specific compounds disclosed herein containing ⁶³Zn, the present disclosure also embraces those generic structures or specific compounds with a non-radioactive zinc isotope, such as ⁶⁴Zn, ⁶⁶Zn, ⁶⁷Zn, ⁶⁸Zn, or ⁷⁰Zn, or with zinc at natural isotopic abundance (approximately ⁶⁴Zn (48.6%), ⁶⁶Zn (27.9%), ⁶⁷Zn (4.1%), ⁶⁸Zn (18.8%), and ⁷⁰Zn (0.6%)), or with ⁶⁴Zn, ⁶⁶Zn, ⁶⁷Zn, ⁶⁸Zn, and ⁷⁰Zn in any ratio. For any of the generic structures or specific compounds disclosed herein containing ⁵²Mn, the present disclosure also embraces those generic structures or specific compounds with a non-radioactive Mn isotope, such as ⁵⁵Mn. In further embodiments, the disclosure provides any of the NSAID derivative compounds disclosed herein, with the removal of the metal group or radioactive agent. Thus, for any of the generic structures or specific conjugates disclosed herein containing copper, such as ⁶³Cu, ⁶⁴Cu, ⁶⁵Cu, copper at natural isotopic abundance, copper isotopes in any ratio, or their oxides or carbonyl derivatives, or any of the generic structures or specific conjugates disclosed herein containing gallium, such as ⁶⁸Ga, ⁶⁹Ga, ⁷¹Ga, gallium at natural isotopic abundance, or gallium isotopes in any ratio, or any of the generic structures or specific conjugates disclosed herein containing zinc, such as ⁶³Zn, ⁶⁴Zn, ⁶⁶Zn, ⁶⁷Zn, ⁶⁸Zn, or ⁷⁰Zn, zinc at natural isotopic abundance, or zinc isotopes in any ratio, or any of the generic structures or specific conjugates disclosed herein containing manganese, such as ⁵²Mn or ⁵⁵Mn, or Mn isotopes in any ratio, the disclosure also embraces those generic structures or specific conjugates without the metal or metal derivative, that is, with the uncomplexed (free) chelator.

In further embodiments, the present disclosure provides any of the NSAID derivative compounds disclosed herein, with the substitution of a different copper isotope for the copper isotope shown in the structure. Thus, for any of the generic structures or specific compounds disclosed herein containing copper, such as ⁶³Cu, ⁶⁴Cu, ⁶⁵Cu, copper at natural isotopic abundance, copper isotopes in any ratio, or their oxides or carbonyl derivatives, the present disclosure also embraces those generic structures or specific compounds with a different copper isotope selected from ⁶³Cu, ⁶⁴Cu, ⁶⁵Cu, copper at natural isotopic abundance, copper isotopes in any ratio, or their oxides or carbonyl derivatives. For any of the generic structures or specific compounds disclosed herein containing gallium, such as ⁶⁸Ga, ⁶⁹Ga, ⁷¹Ga, gallium at natural isotopic abundance, or gallium isotopes in any ratio, the present disclosure also embraces those generic structures or specific compounds with a different gallium isotope selected from ⁶⁸Ga, ⁶⁹Ga, ⁷¹Ga, gallium at natural isotopic abundance, or gallium isotopes in any ratio. For any of the generic structures or specific conjugates disclosed herein containing zinc, such as ⁶³Zn, ⁶⁴Zn, ⁶⁶Zn, ⁶⁷Zn, ⁶⁸Zn, or ⁷⁰Zn, zinc at natural isotopic abundance, or zinc isotopes in any ratio, the present disclosure also embraces those generic structures or specific compounds with a different zinc isotope selected from ⁶³Zn, ⁶⁴Zn, ⁶⁶Zn, ⁶⁷Zn, ⁶⁸Zn, or ⁷⁰Zn, zinc at natural isotopic abundance, or zinc isotopes in any ratio. For any of the generic structures or specific conjugates disclosed herein containing manganese, such as ⁵²Mn, ^{52m}Mn, ^{52g}Mn, or ⁵⁵Mn, the present disclosure also embraces those generic structures or specific compounds with a different manganese isotope selected from ⁵²Mn, ^{52m}Mn, ^{52g}Mn, or ⁵⁵Mn.

In further embodiments, the disclosure provides any of the NSAID derivative compounds disclosed herein used with a combination of different metals, whether radioactive or non-radioactive. For example, any of the NSAID derivative compounds disclosed herein can be used as a mixture of one or more NSAID derivative compounds with ⁶⁴Cu and one or more NSAID derivative compounds with ⁶⁸Ga in any ratio; a mixture of one or more NSAID derivative compounds with ⁶⁴Cu and one or more NSAID derivative compounds with ⁶³Zn in any ratio; a mixture of one or more NSAID derivative compounds with ⁶⁸Ga and one or more NSAID derivative compounds with ⁶³Zn in any ratio; or a mixture of one or more NSAID derivative compounds with ⁶⁴Cu, one or more NSAID derivative compounds with ⁶⁸Ga, and one or more NSAID derivative compounds with ⁶³Zn in any ratio. ⁵²Mn, ^{52m}Mn, or ^{52g}Mn can also be used with any or all of the foregoing combinations.

In further embodiments, the disclosure provides the synthesis of any of the NSAID derivative compounds described herein, according to the protocols disclosed herein.

In further embodiments, the disclosure provides the compound:

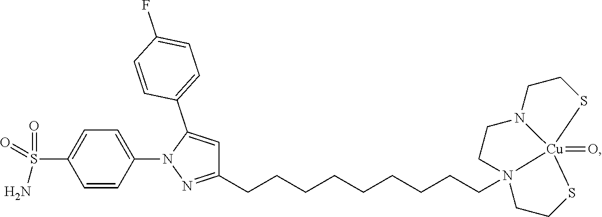

or a salt thereof.

In further embodiments, the disclosure provides the compound:

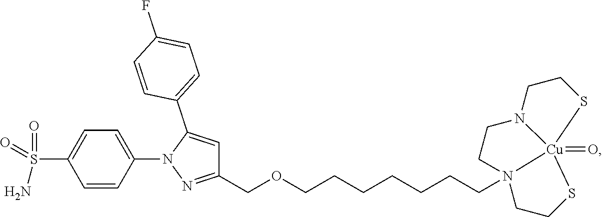

or a salt thereof.

In further embodiments, the disclosure provides the compound:

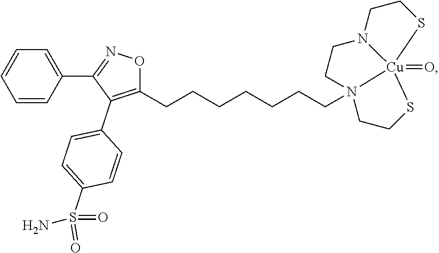

or a salt thereof.

In further embodiments, the disclosure provides the compound:

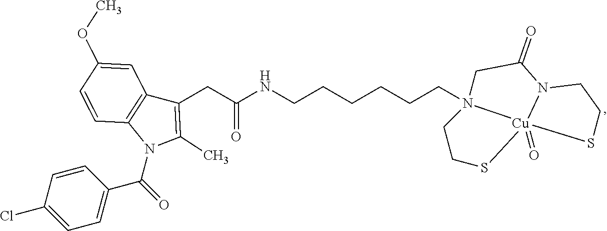

or a salt thereof.

In further embodiments, the disclosure provides the compound:

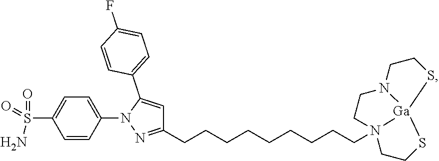

or a salt thereof.

In further embodiments, the disclosure provides the compound:

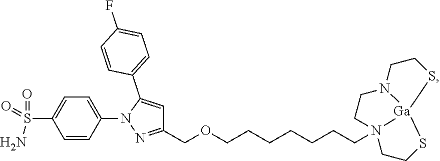

or a salt thereof.

In further embodiments, the disclosure provides the compound:

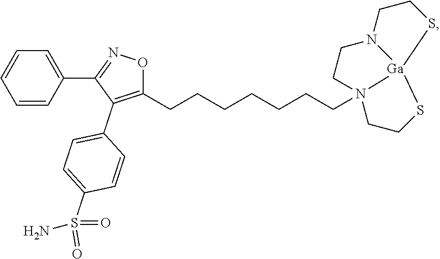

or a salt thereof.

In further embodiments, the disclosure provides the compound:

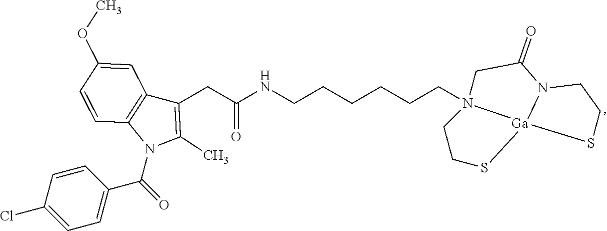

or a salt thereof.

In further embodiments, the disclosure provides the compound:

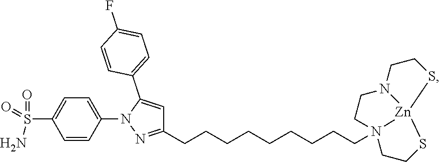

or a salt thereof.

In further embodiments, the disclosure provides the compound:

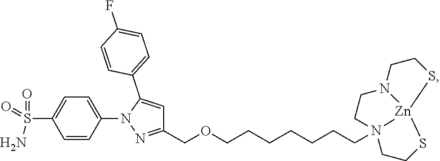

or a salt thereof.

In further embodiments, the disclosure provides the compound:

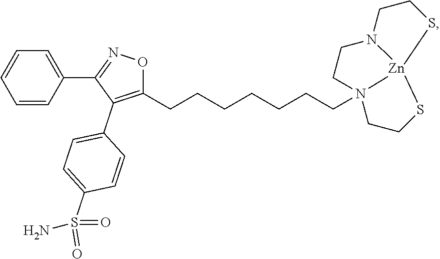

or a salt thereof.

In further embodiments, the disclosure provides the compound:

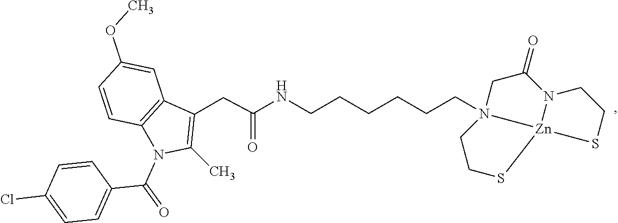

or a salt thereof.

In further embodiments, the disclosure provides the compound:

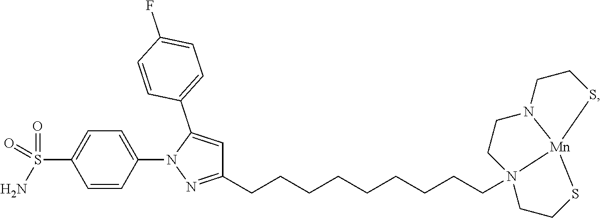

or a salt thereof.

In further embodiments, the disclosure provides the compound:

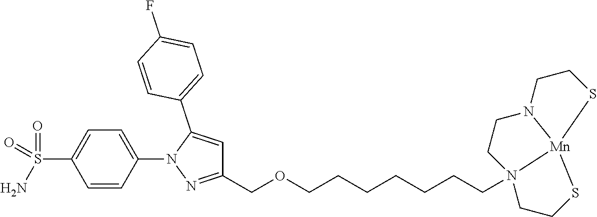

or a salt thereof.

In further embodiments, the disclosure provides the compound:

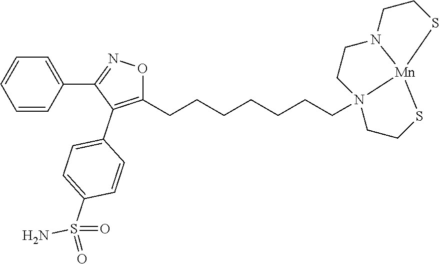

or a salt thereof.

In further embodiments, the disclosure provides the compound:

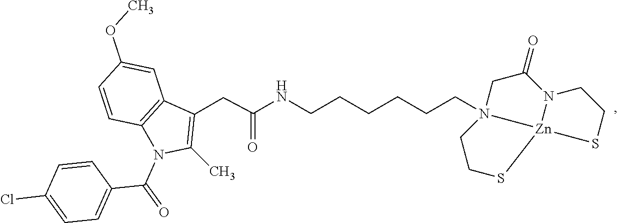

or a salt thereof.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition is described as “consisting essentially of” the listed components, the composition contains the components expressly listed, and may contain other components which do not substantially affect the condition being treated. That is, the composition either does not contain any other components which do substantially affect the condition being treated other than those components expressly listed; or, if the composition does contain extra components other than those listed which substantially affect the condition being treated, the composition does not contain a sufficient concentration or amount of those extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed.

The features of each embodiment disclosed herein are combinable with any of the other embodiments where appropriate and practical.

### DETAILED DESCRIPTION OF THE INVENTION

Identifying sites of pathology is important for proper diagnosis and treatment of a patient. However, it can often be difficult to pinpoint the precise location of pathology. Extensive imaging and testing may be required to accurately identify the source of pathology.

Tumor localization is an example of a condition where it can be difficult to precisely identify the area of pathology, e.g., in a patient with metastatic adenocarcinoma who presents with clear metastasis, but where the primary site of the malignancy is not known. The secondary sites of the tumor (metastases) are difficult to find in many cancer cases. This problem also occurs with “benign tumors” such as giant cell tumors, which rarely metastasize, and “quasi-malignant tumors” such as adamantinomas, which rarely metastasize early, but are known to metastasize late in their course. Because the tumor location can be extremely difficult to find, a new test which could reveal all types of tumor cells would facilitate tumor searches, whether primary tumor sites or metastatic tumor sites, and help determine the appropriate treatment.

Pain is a common symptom in medicine and is another condition where the source of the pathology is not always readily apparent, despite thorough physical exams, laboratory studies, and radiologic studies and analysis. This is especially true for low back pain and abdominal pain. Pain in the body results from various compounds produced and released at the site of the injured area. These pain-producing compounds include bradykinins, prostaglandins, chemokines, histamine, and others. Importantly, the site at which the patient perceives the pain may not be the site of the actual injury or pathology. The term “referred pain” is used to describe pain that is perceived by the patient at a site distinct from the pathology. Referred pain can complicate diagnosis, location of the actual site of pathology, and determination of appropriate treatment. Imaging of patients using the compounds disclosed herein can locate the site of pathology that causes pain, such as back pain, abdominal pain, and neck pain.

Prostaglandins, especially the PG₂ group of prostaglandins, are over-expressed in tumor cells. Prostaglandins (such as the PG₂ group of prostaglandins) are also strongly associated with the experience of pain. Because prostaglandins are produced at the site of tumor location, actual injury, or pathology, identifying the site where prostaglandin synthesis occurs will assist in locating the precise area of pathology. Biosynthesis of the PG₂ prostaglandins requires the cyclooxygenase (COX) enzyme. The cyclooxygenase enzyme exists in (at least) two isoforms, COX-1, which is expressed constitutively, but which may be upregulated at sites of pain and inflammation, and COX-2, which is inducible by inflammatory stimuli. Both COX-1 and COX-2 are upregulated at tumor sites. Areas of high expression of cyclooxygenase will be associated with areas of high production of prostaglandins, which in turn are associated with the area of pathology. Thus, pinpointing areas of high cyclooxygenase expression will enable identification of the pathological area.

The cyclooxygenase enzymes are readily inhibited by non-steroidal anti-inflammatory drugs (NSAIDs), which are sold over the counter in most countries, and also often frequently prescribed by doctors. These non-steroidal anti-inflammatory medicines include several pharmaceutical classes; each class has a number of specific drugs. If a NSAID drug is bonded or complexed to an imaging moiety, partial or total imaging of the patient provides a method of identifying sites of cyclooxygenase overexpression, prostaglandin synthesis, and inflammation, which determines the site of pathology or injury. Thus, in one embodiment, the current disclosure provides NSAID derivative compounds which have a residue or fragment of the NSAID valdecoxib, the NSAID celecoxib, or the NSAID indomethacin; an imaging moiety; and a linker joining the residue or fragment of the NSAID with the imaging moiety. The NSAID derivative compound is suitable for imaging with an appropriate imaging modality.

In addition to NSAID derivative compounds suitable for imaging, the disclosure also provides NSAID derivative compounds which are not used for imaging, but which are useful surrogates for studying the chemical, biological, and pharmacokinetic properties of the compounds suitable for imaging. Substitution of non-radioactive isotopes of copper (such as ⁶³Cu, ⁶⁵Cu, or copper at natural isotopic abundance) for ⁶⁴Cu results in a compound which can be handled without the need for radiation protection. Substitution of non-radioactive isotopes of gallium (such as ⁶⁹Ga, ⁷¹Ga, or gallium at natural isotopic abundance) for ⁶⁸Ga results in a compound which can be handled without the need for radiation protection. Substitution of non-radioactive isotopes of zinc (such as ⁶⁴Zn, ⁶⁶Zn, ⁶⁷Zn, ⁶⁸Zn, ⁷⁰Zn, or zinc at natural isotopic abundance) for ⁶³Zn results in a compound which can be handled without the need for radiation protection. Substitution of non-radioactive isotopes of manganese (such as ⁵⁵Mn) for ⁵²Mn results in a compound which can be handled without the need for radiation protection. Accordingly, preparation of compounds which have non-radioactive copper isotopes in place of radioactive copper isotopes, non-radioactive gallium isotopes in place of radioactive gallium isotopes, non-radioactive zinc isotopes in place of radioactive zinc isotopes, or non-radioactive manganese isotopes in place of radioactive manganese isotopes, can be useful for chemical, physical, in vitro, and in vivo studies of compound properties in which the imaging properties of the compound are not under study, such as studies of toxicity and biological half-life, and the disclosure provides both the compounds suitable for imaging and their analogs which can be handled without radiation precautions.

The NSAID derivative compounds are also useful for diagnosis of infections. Infections cause cells to overexpress the COX-1 and COX-2 enzymes. The pattern distribution of the cellular influence for the three major types of infections, bacterial, tuberculosis (TB), or viral, differ in major ways. Bacterial infections (not including TB) affect COX production in the cells of most of the body's organs. The compounds disclosed herein can be used for diagnosis of any bacterial infection, and are particularly useful in abscess forming bacteria, in subjects or patients with an organ-specific infection, and in aiding in diagnosis and determination of the cause of a fever of unknown origin (FUO). The organ most involved would produce more COX enzyme than the rest of the body's tissues, even though all tissues may show some increased activity.

TB infections can infect almost any organ, such as the lungs, the testes, the spinal column (such as psoas abscess), etc. Scans conducted with compounds disclosed herein can help pinpoint the major locus of TB infection, which is especially helpful in a subject or patient with a positive skin reaction to TB (such as a positive PPD test). The primary locus for a TB infection would likely be at the site of the highest gamma count on a gamma camera when a gamma-emitting radioactive moiety is used in the compound.

Viral infections tend to first cause elevated COX production in the spleen to a great extent and in the stomach to a slightly lesser extent. The compounds disclosed herein can thus be used for the screening of asymptomatic patients infected with a virus. Patients are frequently infectious even before they exhibit symptoms, such as patients with Ebola virus and other viruses. An asymptomatic patient or subject who has been exposed to such viruses, such Ebola virus, influenza viruses, corona viruses (including severe acute respiratory syndrome coronavirus 2, or SARS-CoV-2), or other viruses deemed sufficiently important for screening, or who has traveled in areas where outbreaks of such viruses have occurred, can be screened by administration of compounds disclosed herein, followed by imaging. When the NSAID derivative compound comprises a positron-emitting radioactive moiety, positron emission tomography (PET) can detect signals above background, and thus increased COX expression, from at least the spleen and probably the stomach, indicating the presence of an infection.

### Definitions

“Alkyl” is intended to embrace a univalent saturated linear or branched hydrocarbon chain having the number of carbon atoms specified, or if no number is specified, having 1 to 12 carbon atoms, such as 1 to 10 carbon atoms, or 1 to 8 carbon atoms. “Alkylene” refers to a similar group, which is divalent. Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Optionally substituted” alkyl refers to either an unsubstituted alkyl group, or an alkyl group substituted with one or more substituents (such as one, two, three, four, or five substituents) selected from the group consisting of —OH, —(C₁-C₄ alkyl)-OH, halo, fluoro, chloro, bromo, iodo, —(C₁-C₄ alkyl), —(C₁-C₄) haloalkyl, —(C₁-C₄) perhaloalkyl, —O—(C₁-C₄ alkyl), —O—(C₁-C₄ haloalkyl), —O—(C₁-C₄ perhaloalkyl), —(C₁-C₄) perfluoroalkyl, —(C═O)—(C₁-C₄) alkyl, —(C═O)—(C₁-C₄) haloalkyl, —(C═O)—(C₁-C₄) perhaloalkyl, —NH₂, —NH(C₁-C₄ alkyl), —N(C₁-C₄ alkyl)(C₁-C₄ alkyl) (where each C₁-C₄ alkyl is chosen independently of the other), —NO₂, —CN, isocyano (NC—), oxo (═O), —C(═O)H, —C(═O)—(C₁-C₄ alkyl), —COOH, —C(═O)—O—(C₁-C₄ alkyl), —C(═O)NH₂, —C(═)ONH(C₁-C₄ alkyl), —C(═O)N(C₁-C₄ alkyl)(C₁-C₄ alkyl) (where each C₁-C₄ alkyl is chosen independently of the other), —SH, —(C₁-C₄ alkyl)-SH, —S—(C₁-C₄ alkyl), —S(═O)—(C₁-C₄ alkyl), —SO₂—(C₁-C₄ alkyl), and —SO₂—(C₁-C₄ perfluoroalkyl). Examples of such substituents are —CH₃, —CH₂CH₃, —CF₃, —CH₂CF₃, —CF₂CF₃, —OCH₃, —NH(CH₃), —N(CH₃)₂, —SCH₃, and SO₂CH₃. Alternatively, substituents or optional substituents can be specified for a particular group. “Optionally substituted alkylene” groups can be unsubstituted or substituted in the same manner as substituted alkyl groups. It is understood that when alkylene is substituted (for example with a cycloalkyl group), the substituent is not one of the sites of bivalency. For example, propylene substitution with cyclopropyl may provide

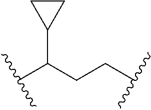 [SMILES: *CCC(*)C1CC1]

but does not provide

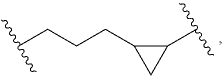 [SMILES: *CCCC1CC1*]

wherein the wavy line denotes a site of bivalency.

“Haloalkyl” is intended to embrace a univalent saturated linear or branched hydrocarbon chain having the number of carbon atoms specified, or if no number is specified, having 1 to 12 carbon atoms, such as 1 to 10 carbon atoms, or 1 to 8 carbon atoms, which bears at least one halogen substituent. “Haloalkylene” refers to a similar group, which is divalent. Particular haloalkyl groups are those having 1 to 20 carbon atoms (a “C₁-C₂₀ haloalkyl”), having 1 to 10 carbon atoms (a “C₁-C₁₀ haloalkyl”), having 6 to 10 carbon atoms (a “C₆-C₁₀ haloalkyl”), having 1 to 6 carbon atoms (a “C₁-C₆ haloalkyl”), having 2 to 6 carbon atoms (a “C₂-C₆ haloalkyl”), or having 1 to 4 carbon atoms (a “C₁-C₄ haloalkyl”). An example of a haloalkyl group is trifluoromethyl, —CF₃. An example of a haloalkylene group is —(CF₂)—(CH₂)₅—. The halogen can be F, Cl, Br, or I, particularly F.

“Cycloalkyl” is intended to embrace a univalent saturated cyclic hydrocarbon chain having the number of carbon atoms specified, or if no number is specified, having 3 to 10 carbon atoms, such as 3 to 8 carbon atoms or 3 to 6 carbon atoms. A cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃-C₈ cycloalkyl”), having 3 to 6 annular carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like. “Cycloalkylene” refers to a similar group, which is divalent. Cycloalkylene can consist of one ring or multiple rings which may be fused, spiro or bridged, or combinations thereof. Particular cycloalkylene groups are those having from 3 to 12 annular carbon atoms. A preferred cycloalkylene is a cyclic hydrocarbon having from 3 to 8 annular carbon atoms (a “C₃-C₈ cycloalkylene”), having 3 to 6 carbon atoms (a “C₃-C₆ cycloalkylene”), or having from 3 to 4 annular carbon atoms (a “C₃-C₄ cycloalkylene”). Examples of cycloalkylene include, but are not limited to, cyclopropylene, cyclobutylene, cyclopentylene, cyclohexylene, cycloheptylene, norbornylene, and the like. A cycloalkylene may attach to the remaining structures via the same ring carbon atom (e.g., 1,1-cyclopropylene) or different ring carbon atoms (e.g., 1,2-cyclopropylene). When a cycloalkylene attaches to the remaining structures via two different ring carbon atoms, the connecting bonds may be cis or trans to each other (e.g., cis-1,2-cyclopropylene or trans-1,2-cyclopropylene). If points of attachment are not specified, the moiety can include any chemically possible attachments. For example, cyclopropylene can indicate 1,1-cyclopropylene or 1,2-cyclopropylene (e.g., cis-1,2-cyclopropylene, trans-1,2-cyclopropylene, or a mixture thereof), or a mixture thereof. Cycloalkyl and cycloalkylene groups can be unsubstituted or substituted in the same manner as substituted alkyl groups where chemically possible.

“Heteroalkyl” is defined as a univalent alkyl group in which at least one carbon atom of the alkyl group is replaced by a heteroatom, such as O, S, or N. Substituents on the third valence in a nitrogen atom in a heteroalkyl group include, but are not limited to, hydrogen or C₁-C₄ alkyl. “Heteroalkylene” refers to a similar group, which is divalent. Examples of heteroalkyl and heteroalkylene groups include, but are not limited to, ethylene glycol and polyethylene glycol moieties, such as (—CH₂CH₂—O)ₙ—H (a monovalent heteroalkyl group) and (—CH₂CH₂—O—)ₙ (a divalent heteroalkylene group) where n is an integer from 1 to 12 inclusive, and propylene glycol and polypropylene glycol moieties, such as (—CH₂CH(CH₃)—O—)ₙ—H (a monovalent heteroalkyl group) and (—CH₂CH(CH₃)—O—)ₙ— (a divalent heteroalkylene group) where n is an integer from 1 to 12 inclusive. Heteroalkyl and heteroalkylene groups can be unsubstituted or substituted in the same manner as substituted alkyl groups where chemically possible.

“Alkenyl” is intended to embrace a univalent linear or branched hydrocarbon chain having at least one carbon-carbon double bond, and having the number of carbon atoms specified, or if no number is specified, having 2 to 12 carbon atoms, such as 2 to 10 carbon atoms or 2 to 8 carbon atoms. An alkenyl group may have “cis” or “trans” configurations, or alternatively have “E” or “Z” configurations. Particular alkenyl groups are those having 2 to 20 carbon atoms (a “C₂-C₂₀ alkenyl”), having 6 to 10 carbon atoms (a “C₆-C₁₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl groups include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, pent-1-enyl, pent-2-enyl, hex-1-enyl, hex-2-enyl, hex-3-enyl, and the like. “Alkenylene” refers to a similar group, which is divalent. Particular alkenylene groups are those having 2 to 20 carbon atoms (a “C₂-C₂₀ alkenylene”), having 2 to 10 carbon atoms (a “C₂-C₁₀ alkenylene”), having 6 to 10 carbon atoms (a “C₆-C₁₀ alkenylene”), having 2 to 6 carbon atoms (a “C₂-C₆ alkenylene”), 2 to 4 carbon atoms (a “C₂-C₄ alkenylene”) or 2 to 3 carbon atoms (a “C₂-C₃ alkenylene”). Examples of alkenylene include, but are not limited to, groups such as ethenylene (or vinylene) (—CH═CH—), propenylene (—CH═CHCH₂—), 1,4-but-1-enylene (—CH═CH—CH₂CH₂—), 1,4-but-2-enylene (—CH₂CH═CHCH₂—), 1,6-hex-1-enylene (—CH═CH—(CH₂)₃CH₂—), and the like. Alkenyl and alkenylene groups can be unsubstituted or substituted in the same manner as substituted alkyl groups where chemically possible.

“Cycloalkenyl” is intended to embrace a univalent cyclic hydrocarbon chain having at least one carbon-carbon double bond and having the number of carbon atoms specified, or if no number is specified, having 4 to 10 carbon atoms, such as 4 to 8 carbon atoms or 4 to 6 carbon atoms. “Cycloalkenylene” refers to a similar group, which is divalent. Cycloalkenyl and cycloalkenylene groups can be unsubstituted or substituted in the same manner as substituted alkyl groups where chemically possible.

“Alkynyl” is intended to embrace a univalent linear or branched hydrocarbon chain having at least one carbon-carbon triple bond, and having the number of carbon atoms specified, or if no number is specified, having 2 to 12 carbon atoms, such as 2 to 10 carbon atoms or 2 to 8 carbon atoms. Particular alkynyl groups are those having 2 to 20 carbon atoms (a “C₂-C₂₀ alkynyl”), having 6 to 10 carbon atoms (a “C₆-C₁₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, and the like. “Alkynylene” refers to a similar group, which is divalent. Particular alkynylene groups are those having 2 to 20 carbon atoms (a “C₂-C₂₀ alkynylene”), having 2 to 10 carbon atoms (a “C₂-C₁₀ alkynylene”), having 6 to 10 carbon atoms (a “C₆-C₁₀ alkynylene”), having 2 to 6 carbon atoms (a “C₂-C₆ alkynylene”), 2 to 4 carbon atoms (a “C₂-C₄ alkynylene”) or 2 to 3 carbon atoms (a “C₂-C₃ alkynylene”). Examples of alkynylene include, but are not limited to, groups such as ethynylene (or acetylenylene) (—C≡—C—), propynylene (—C≡CCH₂—), and the like. Alkynyl and alkynylene groups can be unsubstituted or substituted in the same manner as substituted alkyl groups where chemically possible.

The various groups described above can be attached to the remainder of the molecule at any chemically possible location on the fragment. For the purposes of drawing the structures, groups are typically attached by replacement of a hydrogen, hydroxyl, methyl, or methoxy group on a “complete” molecule to generate the appropriate fragment, and a bond is drawn from the open valence on the fragment to the remainder of the molecule. For example, attachment of the heteroalkyl group —CH₂—O—CH₃ proceeds by removal of a hydrogen from one of the methyl groups of CH₃—O—CH₃, to generate the heteroalkyl fragment —CH₂—O—CH₃, from which a bond is drawn from the open valence to the remainder of the molecule.

A “residue” of a non-steroidal anti-inflammatory drug (NSAID) such as celecoxib, valdecoxib, or indomethacin, referred to as an “NSAID residue” or “residue of a NSAID,” is a portion of the NSAID, where the portion of the NSAID retains its ability to bind to cyclooxygenase. Typically, a residue of a NSAID refers to the portion of the molecule left after removal of a hydrogen, a hydroxyl, a methyl, or a methoxy group from the NSAID. The residue is then bonded or complexed together with an imaging moiety. NSAID residues also include portions of an NSAID that retains its ability to bind to cyclooxygenase, where the portion is further modified by the replacement of a hydrogen with a halogen or a trifluoromethyl group, or by the replacement of a methyl group with a trifluoromethyl group, or by the replacement of a hydroxyl group with a methoxy group. In some embodiments, the residue can be connected to a linker, which linker in turn is attached to an imaging moiety, in order to bond or complex the NSAID residue with the imaging moiety.

Reference to “about” a value or parameter herein includes (and describes) variations that are directed to that value or parameter per se. For example, description referring to “about X” includes description of “X”.

The terms “a” or “an,” as used in herein means one or more, unless the context clearly indicates otherwise.

By “subject,” “individual,” or “patient” is meant an individual organism, preferably a vertebrate, more preferably a mammal, most preferably a human.

The description is intended to embrace all salts of the compounds described herein, as well as methods of using such salts of the compounds. In one embodiment, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, can also be prepared. For lists of pharmaceutically acceptable salts, see, for example, P. H. Stahl and C. G. Wermuth (eds.) “Handbook of Pharmaceutical Salts, Properties, Selection and Use, 2nd Revised Edition” Wiley-VCH, 2011 (ISBN: 978-3-906-39051-2). Several pharmaceutically acceptable salts are also disclosed in Berge S. M. et al., J. Pharm. Sci. 66:1-19, (1977).

The disclosure also encompasses, where chemically possible, all stereoisomers and geometric isomers of the compounds, including diastereomers, enantiomers, and cis/trans (E/Z) isomers. The disclosure also encompasses mixtures of stereoisomers and/or geometric isomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

Unless a specific isotope is indicated, the disclosure encompasses all isotopologues of the compounds disclosed herein, such as, for example, deuterated derivatives of the compounds (where H can be ²H, i.e., D).

The groups represented by

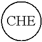

in Formula (I), Formula (II), and Formula (III) are meant to represent metal-binding groups. Groups CHE-1, CHE-2, and CHE-3 are chelating groups with a bound metal, while groups CHE-4, CHE-5 and CHE-6 are chelating groups which do not have a bound metal.

### Linker Position on Valdecoxib, Celecoxib, and Indomethacin Residues

The conjugates of valdecoxib are formed by removing the methyl group at the 5-position of the oxazole ring (circled in the structure below), and using that valence for connection of the linker:

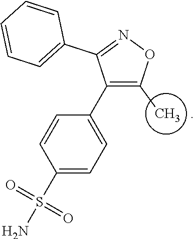 [SMILES: Cc1onc(-c2ccccc2)c1-c1ccc(S(N)(=O)=O)cc1]

The conjugates of celecoxib are formed by removing the trifluoromethyl group at the 3-position of the pyrazole ring (circled in the structure below), and using that valence for connection of the linker:

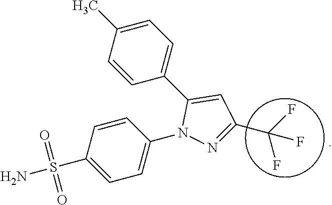 [SMILES: Cc1ccc(-c2cc(C(F)(F)F)nn2-c2ccc(S(N)(=O)=O)cc2)cc1]

The conjugates of indomethacin are formed by removing the acetic acid moiety, and using that opened valence for connection of the linker:

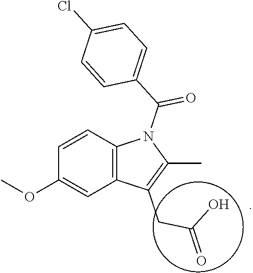 [SMILES: COc1ccc2c(c1)c(CC(=O)O)c(C)n2C(=O)c1ccc(Cl)cc1]

Other modifications of the valdecoxib and celecoxib structures are prepared as indicated by the variable substituents R¹, R², R³, and R⁴ in Formula (I) or Formula (II) as disclosed herein.

### Imaging Compounds Disclosed Herein and Variations Thereof

Provided herein are conjugate compounds, which comprise a non-steroidal anti-inflammatory drug moiety, a linker, and a metal-binding group, which can be a chelating group which can chelate a metal or metal oxide, a cyclopentadienyl group which chelates a metal or metal derivative, or a metallocene group which binds copper. In one embodiment, disclosed herein are conjugate compounds of Formula (I), Formula (II), or Formula (III) as described herein.

In some embodiments, the conjugate compound, or a salt thereof, can have an IC₅₀ for cyclooxygenase inhibition of less than about 0.5 micromolar. The cyclooxygenase can be COX-2.

In further embodiments, disclosed herein is a pharmaceutical composition comprising one or more compounds of any of the conjugate compounds disclosed herein, or a salt thereof, and a pharmaceutically acceptable excipient.

In further embodiments, disclosed here is a method of imaging a site of pathology or suspected pathology in a subject, comprising: a) administering one or more conjugate compounds disclosed herein, or a salt thereof, or a pharmaceutical composition of any of the foregoing, to the subject, wherein M is ⁶⁴Cu, ⁶⁸Ga, ⁶³Zn, or ⁵²Mn, and b) generating an image of the subject or an image of a portion of the subject. The pathology or suspected pathology in the subject can be a tumor or a suspected tumor. The subject can be suffering from pain. The pathology or suspected pathology in the subject can be an infection or a suspected infection.

In further embodiments, disclosed herein is one or more conjugate compounds, or a salt thereof, or a pharmaceutical composition of any of the foregoing, for use in imaging a site of pathology or suspected pathology in a subject. The pathology or suspected pathology in the subject can be a tumor or a suspected tumor. The subject can be suffering from pain. The pathology or suspected pathology in the subject can be an infection or a suspected infection.

In further embodiments, disclosed herein is the use of one or more conjugate compounds disclosed herein, or a salt thereof, or a pharmaceutical composition of any of the forgoing, in the preparation of a medicament for use in imaging a site of pathology or suspected pathology in a subject. The pathology or suspected pathology in the subject can be a tumor or a suspected tumor. The subject can be suffering from pain. The pathology or suspected pathology in the subject can be an infection or a suspected infection. In further embodiments, the present disclosure provides any of the NSAID derivative compounds disclosed herein, with the substitution of a non-radioactive agent for the radioactive agent. Thus, for any of the generic structures or specific compounds disclosed herein containing ⁶⁴Cu, or its oxides or carbonyl derivatives, the present disclosure also embraces those generic structures or specific compounds with non-radioactive Cu, such as ⁶³Cu, ⁶⁵Cu, Cu at natural isotopic abundance (approximately 69.15% ⁶³Cu and 30.85% ⁶⁵Cu), non-radioactive copper isotopes in any ratio, or their oxides or carbonyl derivatives. For any of the generic structures or specific compounds disclosed herein containing ⁶⁸Ga, the present disclosure also embraces those generic structures or specific compounds with non-radioactive Ga, such as ⁶⁹Ga, ⁷¹Ga, Ga at natural isotopic abundance (approximately 60.1% ⁶⁹Ga and 39.9% ⁷¹Ga), or non-radioactive gallium isotopes in any ratio. For any of the generic structures or specific compounds disclosed herein containing zinc, such as ⁶³Zn, ⁶⁴Zn, ⁶⁶Zn, ⁶⁷Zn, ⁶⁸Zn, or ⁷⁰Zn, zinc at natural isotopic abundance, or zinc isotopes in any ratio, the present disclosure also embraces those generic structures or specific compounds with a different zinc isotope selected from ⁶³Zn, ⁶⁴Zn, ⁶⁶Zn, ⁶⁷Zn, ⁶⁸Zn, or ⁷⁰Zn, zinc at natural isotopic abundance, or zinc isotopes in any ratio. For any of the generic structures or specific compounds disclosed herein containing manganese, such as ⁵²Mn, ^{52m}Mn, ^{52g}Mn, or ⁵⁵Mn, the present disclosure also embraces those generic structures or specific compounds with a different manganese isotope selected from ⁵²Mn, ^{52m}Mn, ^{52g}Mn, or ⁵⁵Mn.

In further embodiments, the disclosure provides any of the NSAID derivative compounds disclosed herein, with the removal of the copper. Thus, for any of the generic structures or specific compounds disclosed herein containing ⁶³Cu, ⁶⁴Cu, ⁶⁵Cu, Cu at natural isotopic abundance, copper isotopes in any ratio, or their oxides or carbonyl derivatives, the disclosure also embraces those generic structures or specific conjugates without the metal, that is, with the uncomplexed (free) chelator. For any of the generic structures or specific compounds disclosed herein containing ⁶⁸Ga, ⁶⁹Ga, ⁷¹Ga, Ga at natural isotopic abundance (approximately 60.1% ⁶⁹Ga and 39.9% ⁷¹Ga), or gallium isotopes in any ratio, the disclosure also embraces those generic structures or specific conjugates without the metal, that is, with the uncomplexed (free) chelator. For any of the generic structures or specific conjugates disclosed herein containing zinc, such as ⁶³Zn, ⁶⁴Zn, ⁶⁶Zn, ⁶⁷Zn, ⁶⁸Zn, or ⁷⁰Zn, zinc at natural isotopic abundance, or zinc isotopes in any ratio, the disclosure also embraces those generic structures or specific conjugates without the metal, that is, with the uncomplexed (free) chelator. For any of the generic structures or specific conjugates disclosed herein containing manganese, such as ⁵²Mn, ^{52m}Mn, ^{52g}Mn, or ⁵⁵Mn, the present disclosure also embraces those generic structures or specific conjugates without the metal, that is, with the uncomplexed (free) chelator.

In further embodiments, the disclosure provides the synthesis of any of the NSAID derivative compounds described herein, according to the protocols disclosed herein.

### Cyclooxygenase Binding of the Compounds

The compounds described herein are derivatives of the coxib compounds celecoxib, valdecoxib, or indomethacin. Compounds which can be used for diagnostic and imaging purposes include compounds disclosed herein which have an IC₅₀ for inhibition of a cyclooxygenase, such as COX-2, of less than about 2 micromolar, less than about 1 micromolar less than about 0.5 micromolar, less than about 0.3 micromolar, less than about 0.1 micromolar, less than about 50 nanomolar, or less than about 10 nanomolar.

### Screening for Allergic Reactions

Imaging agents may cause allergic reactions in some patients. In order to screen the NSAID derivative compounds disclosed herein for their potential to cause allergic reactions, the compounds can be screened using tests such as the basophil activation test described in Biological Example G and the ELISA histamine release assay described in Biological Example H. These tests can be used to identify whether compounds have the potential to cause adverse effects, and such compounds can be excluded from further development.

### Formulations and Routes of Administration

The NSAID derivative compounds disclosed herein can be administered in any suitable form that will provide sufficient levels for the purposes of imaging. Intravenous administration is a useful route of administration, although other parenteral routes can also be employed, where parenteral as used herein includes subcutaneous injections, intravenous injection, intraarterial injection, intramuscular injection, intrasternal injection, intraperitoneal injection, or infusion techniques. The compounds can also be administered orally or enterally, which is a preferred route when compatible with the absorption of the compound and with imaging requirements. Where the pharmacokinetics of the compounds are suitable, the compounds can also be administered sublingually, by buccal administration, subcutaneously, by spinal administration, by epidural administration, by administration to cerebral ventricles, by inhalation (e.g. as mists or sprays), rectally (such as by rectal suppository), or topically in unit dosage formulations containing conventional nontoxic pharmaceutically acceptable carriers, excipients, adjuvants, and vehicles as desired. The compounds may be administered directly to a specific or affected organ or tissue. The compounds are mixed with pharmaceutically acceptable carriers, excipients, adjuvants, and vehicles appropriate for the desired route of administration.

In certain embodiments disclosed herein, especially those embodiments where a formulation is used for injection or other parenteral administration, including the routes listed herein, but also including any other route of administration described herein (such as oral, enteric, gastric, etc.), the formulations and preparations used in the methods disclosed herein are sterile. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

Oral administration is advantageous due to its ease of implementation and patient compliance. If a patient has difficulty swallowing, introduction of medicine via feeding tube, feeding syringe, or gastrostomy can be employed in order to accomplish enteric administration. The active compound (and, if present, other co-administered agents) can be enterally administered in any other pharmaceutically acceptable carrier suitable for formulation for administration via feeding tube, feeding syringe, or gastrostomy.

Intravenous administration can also be used advantageously, for delivery of the NSAID derivative compounds disclosed herein to the bloodstream as quickly as possible and to circumvent the need for absorption from the gastrointestinal tract.

The NSAID derivative compounds described for use herein can be administered in solid form, in liquid form, in aerosol form, or in the form of tablets, pills, powder mixtures, capsules, granules, injectables, solutions, suppositories, enemas, colonic irrigations, emulsions, dispersions, food premixes, and in other forms suitable for the route of administration. The compounds can also be administered in liposome formulations. The compounds can also be administered as prodrugs, where the prodrug undergoes transformation in the treated subject to a therapeutically effective form. Additional methods of administration are known in the art.

Injectable preparations, for example, sterile injectable aqueous or oleaginous suspensions, may be formulated according to methods known in the art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono or di-glycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents. Alternatively, the compound may also be administered in neat form if suitable.

The compounds disclosed herein can also be administered in the form of liposomes. As is known in the art, liposomes are generally derived from phospholipids or other lipid substances. Liposomes are formed by mono or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound as disclosed herein, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

The amount of active ingredient that may be combined with the carrier materials to produce a single dosage form can vary depending upon the patient to which the active ingredient is administered and the particular mode of administration. It will be understood, however, that the specific dose level for any particular patient will depend upon a variety of factors including the specific compound employed; the age, body weight, body area, body mass index (BMI), general health, sex, and diet of the patient; the time of administration and route of administration used; the rate of excretion; and the drug combination, if any, used. The compounds can be administered in a unit dosage formulation. The pharmaceutical unit dosage chosen is fabricated and administered to provide sufficient concentration of drug for imaging a patient.

While the compounds disclosed herein can be administered as the sole active pharmaceutical agent, they can also be used in combination with one or more other agents. When additional active agents are used in combination with the compounds disclosed herein, the additional active agents may generally be employed in therapeutic amounts as indicated in the Physicians' Desk Reference (PDR) 53rd Edition (1999), which is incorporated herein by reference, or such therapeutically useful amounts as would be known to one of ordinary skill in the art, or as are determined empirically for each patient.

Combinations of the NSAID derivative compounds can also be used. Combining two or more compounds can provide advantages over using a single compound. Advantages can include the ability to tune pharmacokinetics and pharmacodynamics, to adjust the solubility of the overall composition and/or its components, to adjust the half-life of total compound in the body, to enhance imaging contrast and/or definition, to adjust binding kinetics to COX, to adjust binding affinity to COX, or to enhance the stability of the composition either in storage or in use. The two or more compounds can be combined in solution form such as those solution forms described above (such as in a sterile solution for IV administration), or in solid form such as those solid forms as described above (such as pill or tablet form). The two or more compounds can be mixed together shortly before administration and administered together. The two or more compounds can be administered simultaneously, either by the same route of administration or by different routes of administration. The two or more compounds can be administered consecutively, either by the same route of administration or by different routes of administration. In one embodiment, a kit form can contain two or more compounds as individual compounds, with printed or electronic instructions for administration either as a mixture of compounds, as separate compounds administered simultaneously, or as separate compounds administered consecutively. Where three or more compounds are administered, they can be administered as a mixture of compounds, as separate compounds administered simultaneously, as separate compounds administered consecutively, as separate compounds where two or more may be administered simultaneously with the remainder administered consecutively before or after the simultaneous administration, or any other possible combination of mixed administration, simultaneous administration, and consecutive administration.

### Imaging Techniques

The NSAID derivative compounds comprising the radioactive agent can be used with any suitable imaging technique. ⁶⁴Cu decays by multiple pathways, one of which is positron emission, and thus positron emission tomography can be used with ⁶⁴Cu-containing conjugates. ⁶⁸Ga decays by positron emission, and thus positron emission tomography can be used with ⁶⁸Ga-containing conjugates. ⁶³Zn decays by positron emission, and thus positron emission tomography can be used with ⁶³Zn-containing conjugates. ⁵²Mn decays by positron emission, and thus positron emission tomography can be used with ⁵²Mn-containing conjugates. Exemplary imaging methods that can be used are described in Wernick, M. N. and Aarsvold, J. N., Emission Tomography: The Fundamentals of PET and SPECT, San Diego: Elsevier Academic Press, 2004. Further discussion of copper and gallium complexes as imaging agents is provided in MacPherson D S et al., “A brief overview of metal complexes as nuclear imaging agents,” Dalton Trans. 48(39), 14547-14565, (2019).

Normally, COX-2 expression is not observed in most tissues. Qualitative detection of imaging agents in a specific region is indicative of elevated COX-2 expression levels, that is, elevated levels of COX-2 enzyme. Such qualitative detection is diagnostic of a pain generator site or a site of pathology. The relative amount of COX-2 enzyme present can be determined based on the measured levels of radioactivity from the compounds disclosed herein, providing quantitative information on COX-2 enzyme levels (e.g., using a scale reflecting intensity).

### Imaging Applications

Earlier Diagnosis of Rheumatoid Arthritis: Rheumatoid arthritis (RA) is difficult to diagnose especially in the early stages, as the early symptoms are similar to the symptoms of several other diseases and the sensitivity of current methods is inadequate. As a result, at least 30% of patients are not diagnosed at an early stage that could delay or prevent disease progression and severity. It is well established that an early diagnosis of RA with early intervention leads to better patient outcomes. However, currently there are no blood or imaging tests to confirm or rule out an early diagnosis of RA. Diagnosis of RA is about 70% accurate and may not include the extent of the RA throughout the body. Providing a method for accurate early diagnosis of RA will enable treatment to begin earlier in the disease process, can improve patient outcome, and reduce costs associated with the disease.

Imaging with a compound that binds to COX-2, such as the compounds disclosed herein, can significantly improve the sensitivity of the diagnosis and provide guidance on how wide-spread the disease is. A patient usually presents with extremity pain that is non traumatic and with morning stiffness. Because the constellation of joint involvement in RA is not unique in the early stages of the disease, imaging with compounds, such as those disclosed herein, can be used to rule out other causes of autoimmune disorders, leading to more certainty in a diagnosis of RA. For instance, psoriatic arthritis, ankylosing spondylitis, and Reiter's syndrome can present only with extremity joint pain. However, it also known that these diseases frequently involve the spine, whereas RA does not. If increased binding of an imaging compound, such as the compounds disclosed herein, is noted in the spinal region on the scan then the diagnosis of RA can be eliminated. In addition, any increased uptake in the kidney could signify inflammation of the kidney that is caused by systemic lupus erythematous (SLE nephritis) which, again, eliminates the diagnosis of RA.

Joints which can be affected by rheumatoid arthritis include the proximal interphalangeal and metacarpophalangeal joints of the hands (i.e., the finger joints and knuckles) and the wrist joints. The distal interphalangeal joints may also be affected, although this is less common. Joints in the feet which may be affected include, but are not limited to, the metatarsophalangeal joints. Other joints which may be affected include the shoulders, elbows, knees, and ankles. Any or all of these joints can be imaged with compounds that bind to COX-2, such as with the compounds disclosed herein, for diagnosis, evaluation, and treatment.

Evaluating Efficacy of Treatment of Rheumatoid Arthritis: Patients can be treated for rheumatoid arthritis (RA) using several therapies, including various pharmaceutical agents, physical therapy, or surgery. In the United States, approximately 900,000 RA patients per year are treated with anti-TNF antibodies such as Humira®. These treatments are expensive and carry the risk of side effects such as infection. In addition, approximately 40% of patients treated with anti-TNF antibodies stop responding to the treatment within a year. Early determination of the efficacy and of patient response to treatment can thus avoid both side effects and unnecessary costs of treatment.

Imaging agents for COX-2 enzyme levels, such as the compounds disclosed herein, can be used as a companion diagnostic to identify when antibody treatment has stopped working. Imaging scans with such agents can be used on a regular schedule. If the practitioner sees that the COX-2 enzyme levels are not going down, they can discontinue treatment. This would save expense and reduce the patient side effects of the treatment that is no longer working.

Evaluating Need for Opioid Treatment: Physicians currently do not have an objective quantifiable diagnostic tool to determine if a patient actually has pain that requires opioid treatment. Though states have developed guidelines or suggestions on the proper length of time to utilize opioid therapy, it has not been shown that these guidelines are adequate to reliably guide clinical practice. Imaging with an agent that indicates levels of COX-2 enzyme, such as the compounds disclosed herein, represents a more objective method for determining the necessity of opioids.

Opioid misuse is a severe problem in the United State and in other countries, underscoring the importance of ensuring that patients with severe pain are appropriately treated, and also that patients that do not need opioid drugs to control pain are appropriately excluded from opioid treatment. In the United States, over 190 million opioid prescriptions are written per year. The United States is in the midst of an opioid crisis which began because of the significant misuse of prescription opioids. Four out of five heroin users began using heroin after using prescription opioids, underscoring the need for determining when opioid drugs are truly needed.

Pain physicians and primary care doctors do not have an objective and quantifiable way of deciding on writing a prescription for opioids. Imaging with an agent that indicates levels of COX-2 enzyme, such as the compounds disclosed herein, can provide important information on the levels of COX 2 enzyme in the body. If elevated COX-2 is not seen on exam, then an opioid prescription is not indicated. Imaging with agents such as those disclosed herein can play a significant role in reducing the number of prescriptions, while making sure that the patients that truly need opioids are appropriately taken care of.

Evaluating Suitability for Treatment with Anti-Nerve Growth Factor Antibodies: Anti-nerve growth factor antibodies have been proposed as a treatment for pain, such as chronic low back pain. However, anti-NGF antibody treatment has also been associated with adverse effects such as joint damage (see, e.g., Markman, J. D. et al., Pain 161 (2020) 2068-2078). Screening patients beforehand for elevated levels of COX-2 expression in joints can identify patients who should be excluded from anti-NGF therapy. For example, a patient with elevated COX-2 expression in one or more joints can be excluded from anti-NGF therapy, while patients without elevated COX-2 expression in a joint need not be excluded on that basis.

### Kits

Further embodiments of the disclosure provide one or more kit forms which can contain one or more NSAID derivative compounds as disclosed herein. The kit can contain printed or electronic instructions for administration of the one or more compounds. In further embodiments, the kit can contain one or more compounds as disclosed herein which lacks the radioactive agent with printed or electronic instructions for adding the radioactive agent to constitute one or more compounds disclosed herein.

The following examples are intended to illustrate, but not limit, the disclosure.

### EXAMPLES

### Synthetic Examples

The following abbreviations may be used herein:
˜ about
+ve or pos. ion positive ion
Δ heat
Ac Acetyl
ACN acetonitrile
Ac₂O acetic anhydride
aq aqueous
AcOH acetic acid
Bn benzyl
Boc tert-butyloxycarbonyl
Bu butyl
Bz benzoyl
Calcd or Calc'd calculated
Conc. concentrated
Cp cyclopentadiene
d day(s) or (NMR) doublet (NMR)
dd doublet of doublets (NMR)
D5W 5% dextrose solution in water
DCE dichloroethane
DCM dichloromethane
DEA diethylamine
DIEA or DIPEA diisopropylethylamine
DMAP 4-dimethylaminopyridine
DME 1,2-dimethoxyethane
DMF N,N-dimethylformamide
DMSO dimethyl sulfoxide
EDC or EDCI N-ethyl-N′-(3-dimethylaminopropyl)carbodiimide
eq equivalent
ESI or ES electrospray ionization
Et ethyl
Et₂O diethyl ether
Et₃N triethylamine
EtOAc or EA ethyl acetate
EtOH ethyl alcohol
FA formic acid
g gram(s)
h hour(s)
Hex hexanes
HOBT hydroxybenzotriazole
HPLC high pressure liquid chromatography
IPA or iPrOH isopropyl alcohol
KOAc potassium acetate
LCMS, LC-MS or LC/MS liquid chromatography mass spectrometry
LDA lithium diisopropylamide
LHMDS or LiHMDS lithium hexamethyldisilazide
M molar (mol L⁻¹)
Me methyl
MeCN acetonitrile
Mel iodomethane
MeOH methyl alcohol
mg milligram(s)
min minute(s)
mL milliliter(s)
M mole(s)
MS mass spectrometry
MsCl methanesulfonyl chloride
MTBE or MtBE methyl tert-butyl ether
m/z mass-to-charge ratio
NaHMDS sodium hexamethyldisilazide
NaOtBu sodium tert-butoxide
NBS N-bromosuccinimide
nBuLi n-butyl lithium
NMO N-methylmorpholine-N-oxide
NMP 1-methyl-2-pyrrolidinone
NMR nuclear magnetic resonance
PG(s) prostaglandin(s)
PBS phosphate buffered saline
PMB paramethoxybenzyl
Pr propyl
ppm parts per million
PTFE polytetrafluoroethylene
p-tol para-toluoyl
rac racemic
RP-HPLC or RPHPLC reversed phase high pressure liquid chromatography
RT or rt or r.t. room temperature
sat. or sat'd or satd saturated
TBDMS tert-butyldimethylsilyl
TBDMS-Cl tert-butyldimethylsilyl chloride
TEA triethylamine
tert or t tertiary
TFA or TFAA trifluoroacetic acid
THE tetrahydrofuran
TLC thin layer chromatography
TMS trimethylsilyl or trimethylsilane
Tr triphenylmethyl
tR retention time
tBuOH tert-butyl alcohol
v/v volume per volume

### Synthesis of Intermediate 1

### N-(2-(Tritylthio)ethyl)-2-((2-(tritylthio)ethyl)amino)acetamide

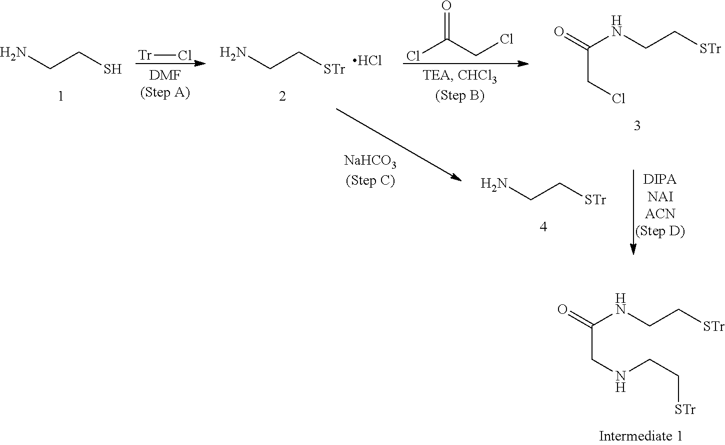

### Step A.

A mixture of cystamine·HCl, compound 1 (22.4 g, 196.8 mmol) and trityl chloride (50 g, 173.1 mmol) in DMF (170 mL) was stirred at rt for 22 h. The reaction mixture was slowly added to ice-cold water (1.5 L) with vigorous stirring. The suspension was allowed to stir for 10 min and then filtered. The precipitate was washed with water (200 mL) and ACN (150 mL). The solids were air dried under vacuum to give 2-(tritylthio)ethan-1-amine hydrochloride, compound 2 (61.5 g 100%) as a white solid.

### Step B.

To a stirred solution of 2-(tritylthio)ethan-1-amine hydrochloride, compound 2 (30.0 g, 84.29 mmol) and triethylamine (30 mL, 210.7 mmol) in chloroform (300 ml) was added a solution of chloroacetyl chloride (30 mL, 84.29 mmol) in dry chloroform (24 ml) slowly over a period of 1 h at 0° C. After addition, the cooling bath was removed and stirring continued for 1 h at rt. The reaction mixture was diluted with DCM and the organic phase was washed with water, sat. aq. NaHCO₃ solution, and brine, dried over Na₂SO₄ and filtered. The filtrate was concentrated in vacuo to give compound 3 (18.0 g, 70%) as amber residue which was pure and used as is in the next step.

### Step C.

40 g of compound 2 was suspended in sat. aq. NaHCO₃ solution (200 mL) and extracted with chloroform (3×150 mL). The combined organic layers were dried over Na₂SO₄, filtered, and the filtrate was concentrated to give the free base 4 as a white solid.

### Step D.

To a stirred suspension of 3 (65.7 g, 166.3 mmol) in ACN (1700 mL) was added 4 (63.7 g, 199.6 mmol), DIPEA (64.51 g, 499 mmol) and NaI (24.95 g, 166.3 mmol), and the reaction was allowed to stir at rt for 72 h. Solvent was evaporated and the residue taken into water 250 mL and extracted with EA (3×200 mL). The organic layers were pooled, washed with sat. aq. NaHCO₃ solution and brine to give the crude Intermediate 1 (50 g) as amber residue. The residue was purified by column chromatography on silica gel eluting with EtOAc/Heptane (40% to 55% to 70%) to give N-(2-(tritylthio)ethyl)-2-((3-(tritylthio)propyl)amino)acetamide, Intermediate 1.

### Synthesis of Intermediate 2

### tert-Butyl (2-(tritylthio)ethyl)(2-((2-(tritylthio)ethyl)amino)ethyl)carbamate

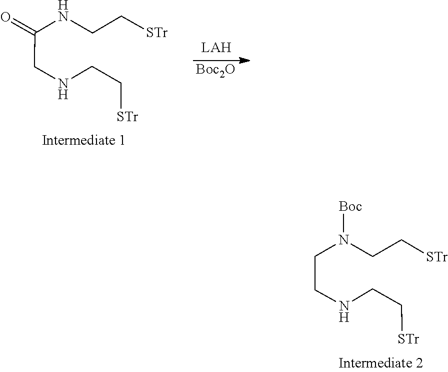

Intermediate 1 is reduced with LiAlH₄ and Boc-protected to give Intermediate 2 according to the procedure described in Ono, M., et al., ACS Chem. Neurosci., 1, 598-607, (2010).

### Example S-01

### Compound 1

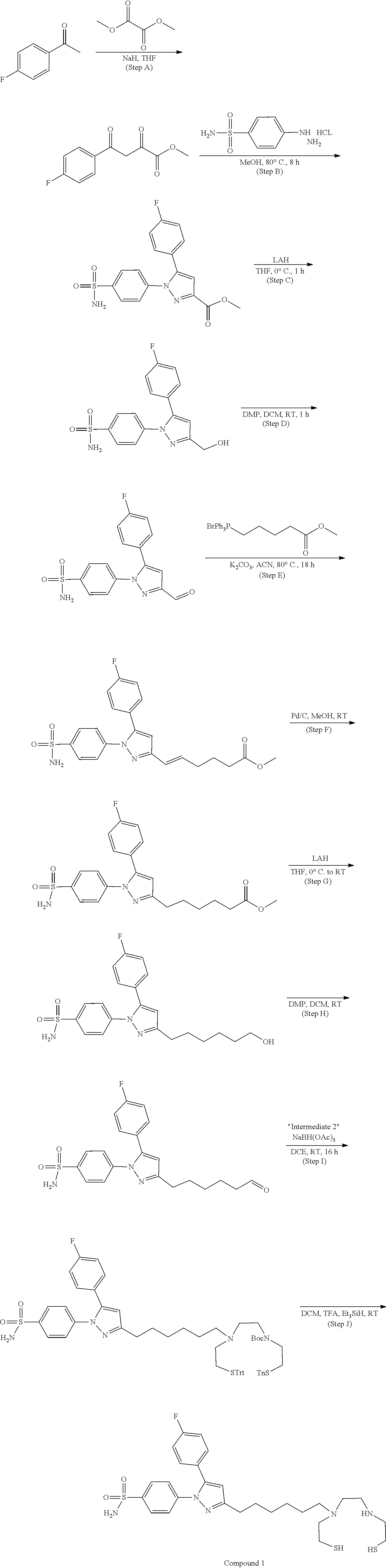

### Step A.

To a solution of 1-(4-fluorophenyl)ethan-1-one (19.3 g, 0.14 mol) in dry THF (0.5 L) was added NaH (11.2 g, 60% dispersion in mineral oil, 0.28 mol) in batches at 0° C. under N₂. After completion, the reaction was stirred at 0° C. for another 30 min. Dimethyl oxalate (17.7 g, 0.15 mol) in THF (200 mL) was added, the resulting mixture was warmed to rt and stirring was continued for 4 h. The reaction was quenched with HCl (1 N aq.) and the pH of the reaction mixture was adjusted to pH=5. The reaction mixture was then extracted with EtOAc (1 L×2). The combined organic layers were dried over Na₂SO₄ and concentrated in vacuo to give methyl 4-(4-fluorophenyl)-2,4-dioxobutanoate (22 g, yield: 70%) as a yellow solid, which was used in the next step without further purification. Mass Spectrum (ESI) m/z=225 (M+1).

### Step B.

A mixture of methyl 4-(4-fluorophenyl)-2,4-dioxobutanoate (11.2 g, 0.050 mol) and 4-hydrazineylbenzenesulfonamide hydrochloride (12.3 g, 0.055 mol) in MeOH (100 ml) was stirred at 80° C. for 3 h. The reaction was cooled to room temperature slowly and filtered. The filter cake was dried under reduced pressure to give methyl 5-(4-fluorophenyl)-1-(4-sulfamoylphenyl)-1H-pyrazole-3-carboxylate (17.0 g, yield: 90%) as a yellow solid, which was used in the next step without purification. Mass Spectrum (ESI) m/z=376 (M+1).

### Step C.

To a solution of methyl 5-(4-fluorophenyl)-1-(4-sulfamoylphenyl)-1H-pyrazole-3-carboxylate (17.0 g, 0.045 mol) in dry THE (0.75 L) was added LiAlH₄ (3.4 g, 0.090 mol) slowly at 0° C. After the reaction was stirred at 0° C. for 1 h, the reaction was quenched with Na₂SO₄ 10H₂O (5.0 g). The resulting mixture was filtered through a plug of Celite© (J. T. Baker, Phillipsberg, NJ, diatomaceous earth) and the filter cake was washed with THE (500 mL). The filtrate was concentrated and purified by column chromatography on silica gel, eluting with 1˜10% MeOH in DCM to give 4-(5-(4-fluorophenyl)-3-(hydroxymethyl)-1H-pyrazol-1-yl)benzenesulfonamide (10.5 g, yield: 67%) as a yellow solid. Mass Spectrum (ESI) m/z=348 (M+1).

### Step D.

To a solution 4-(5-(4-fluorophenyl)-3-(hydroxymethyl)-1H-pyrazol-1-yl) benzene sulfonamide (5.0 g, 14.4 mmol) in DCM (100 mL) was added Dess-Martin periodinane (DMP) (12.2 g, 28.8 mmol) slowly at 0° C. After the resulting mixture was stirred at room temperature for 1 h, the reaction was quenched with sat aq. Na₂S₂O₃ solution (50 mL), followed by sat aq. NaHCO₃ solution (50 ml) and then extracted with DCM (100 mL×3). The combined organic layers were washed with brine, dried over Na₂SO₄, filtered and the filtrate was concentrated under reduced pressure to give the crude product, which was purified by column chromatography on silica gel, eluting with 10-50% EtOAc in PE to give 4-(5-(4-fluorophenyl)-3-formyl-1H-pyrazol-1-yl) benzenesulfonamide (3.0 g, yield: 60%) as a yellow solid. Mass Spectrum (ESI) m/z=346 (M+1).

### Step E.

To a mixture of 4-(5-(4-fluorophenyl)-3-formyl-1H-pyrazol-1-yl)benzenesulfonamide (3.0 g, 8.7 mmol) and K₂CO₃ (3.6 g, 26.1 mmol) in ACN (50 mL) was added (5-methoxy-5-oxopentyl)triphenylphosphonium bromide (5.2 g, 11.3 mmol) at room temperature. After the reaction mixture was stirred at 80° C. for 16 h, the reaction was cooled to room temperature and filtered. The filter cake was washed with ACN (100 mL), the filtrate was concentrated and purified by column chromatography on silica gel, eluting with 10˜50% EtOAc in PE to give methyl 6-(5-(4-fluorophenyl)-1-(4-sulfamoylphenyl)-1H-pyrazol-3-yl)hex-5-enoate (2.2 g, yield: 58%) as a brown solid. Mass Spectrum (ESI) m/z=444 (M+1).

### Step F.

A mixture of methyl 6-(5-(4-fluorophenyl)-1-(4-sulfamoylphenyl)-1H-pyrazol-3-yl)hex-5-enoate (2.2 g, 5.0 mmol) and Pd/C (200 mg) in MeOH (50 mL) was stirred at room temperature for 1 h under H₂. The mixture was filtered, and filter cake was washed with MeOH (30 mL). The filtrate was concentrated in vacuo, the residue was purified by column chromatography on silica gel, eluting with 10˜50% EtOAc in PE to give methyl 6-(5-(4-fluorophenyl)-1-(4-sulfamoylphenyl)-1H-pyrazol-3-yl)hexanoate (1.5 g, yield: 68%) as a brown solid. Mass Spectrum (ESI) m/z=446 (M+1).

### Step G.

To a solution of ethyl methyl 6-(5-(4-fluorophenyl)-1-(4-sulfamoylphenyl)-1H-pyrazol-3-yl)hexanoate (1.5 g, 3.4 mmol) in dry THE (50 mL) was added LiAlH₄ (181 mg, 4.8 mmol) slowly at 0° C. After the mixture was stirred at room temperature for 1 h, the mixture was quenched by Na₂SO₄·10H₂O (2 g). The resulting suspension was filtered through a plug of Celite® (J. T. Baker, Phillipsberg, NJ, diatomaceous earth) and the filter cake was washed with THE (100 mL). The filtrate was concentrated in vacuo, the residue was purified by column chromatography on silica gel, eluting with 10˜50% EtOAc in PE to give 4-(5-(4-fluorophenyl)-3-(6-hydroxyhexyl)-1H-pyrazol-1-yl)benzenesulfonamide (0.8 g, yield: 57%) as a brown solid. Mass Spectrum (ESI) m/z=418 (M+1).

### Step H.

To a solution 4-(5-(4-fluorophenyl)-3-(6-hydroxyhexyl)-1H-pyrazol-1-yl)benzenesulfonamide (0.8 g, 1.9 mmol) in DCM (50 mL) was added Dess-Martin periodinane (DMP) (1.6 g, 3.8 mmol) slowly at 0° C. After the reaction mixture was stirred at room temperature for 1 h, the reaction was quenched by Na₂S₂O₃ (sat. aq., 50 mL), followed by NaHCO₃ (sat., aq., 50 mL) and then extracted with DCM (100 mL×3). The combined organic layers were washed with brine, dried over Na₂SO₄ and concentrated in vacuo to give 4-(5-(4-fluorophenyl)-3-(6-oxohexyl)-1H-pyrazol-1-yl)benzenesulfonamide as a yellow solid (1 g, crude, 60% purity). Mass Spectrum (ESI) m/z=416 (M+1).

### Step I.

To a solution of 4-(5-(4-fluorophenyl)-3-(6-oxohexyl)-1H-pyrazol-1-yl)benzene sulfonamide (1 g, crude from last step) in DCE (20 mL) was added tert-butyl (2-(tritylthio)ethyl)(2-((2-(tritylthio)ethyl)amino)ethyl)carbamate (0.91 g, 1.2 mmol) and 2 drops of CH₃COOH. After the reaction was stirred at rt for 0.5 h, NaBH(OAc)₃ (1.3 g, 6.0 mmol) was added and the reaction was stirred at rt for 16 h. Water (30 mL) was added and the mixture was extracted with DCM (50 mL×3). The combined organic layers were washed with brine, dried over Na₂SO₄, filtered and the filtrate was concentrated. The crude product was purified by column chromatography on silica gel, eluting with 10˜50% EtOAc in PE to give tert-butyl (2-((6-(5-(4-fluorophenyl)-1-(4-sulfamoylphenyl)-1H-pyrazol-3-yl)hexyl)(2-(tritylthio)ethyl)amino)ethyl)(2-(tritylthio)ethyl)carbamate as a white solid (0.4 g, yield: 28%). Mass Spectrum (ESI) m/z=1165 (M+1).

### Step J.

To a solution of tert-butyl (2-((6-(5-(4-fluorophenyl)-1-(4-sulfamoylphenyl)-1H-pyrazol-3-yl)hexyl)(2-(tritylthio)ethyl)amino)ethyl)(2-(tritylthio)ethyl)carbamate (0.4 g, 0.34 mmol) in DCM/TFA (2:1, 6 mL) was added a solution of triethylsilane (39 mg, 0.34 mmol) in DCM (1 mL) slowly at 0° C. After the reaction was stirred at rt for 1 h, the reaction was concentrated in vacuo to give 4-(5-(4-fluorophenyl)-3-(6-((2-mercaptoethyl)(2-((2-mercaptoethyl)amino)ethyl)amino) hexyl)-1H-pyrazol-1-yl)benzenesulfonamide (0.2 g, crude, 60% purity) as yellow oil, to give Compound 1. Mass Spectrum (ESI) m/z=580 (M+1).

Compounds 2-11 were also prepared by procedures similar to the one described in Example S-01, replacing 1-(4-fluorophenyl)ethan-1-one used in Step A and/or (5-methoxy-5-oxopentyl)triphenylphosphonium bromide used in Step E with the reagents shown in Table 1 below.

TABLE 1

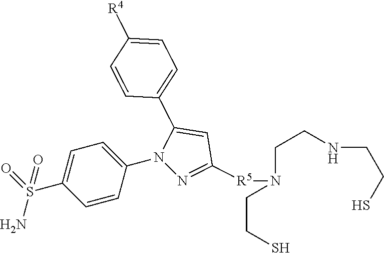

| | | | Reagent used in | Reagent used |
|---|---|---|---|---|
| Compound | R⁴ | R⁶ | Step A | in Step E |
| 2 | F | (CH₂)₅ | 1-(4-fluorophenyl) | methyl 4- |
| | | | ethan-1-one | (bromotriphenyl- |
| | | | | phosphaneyl) |
| | | | | butanoate |
| 3 | F | (CH₂)₇ | 1-(4-fluorophenyl) | methyl methyl 6- |
| | | | ethan- | (bromotriphenyl- |
| | | | 1-one | phosphaneyl) |
| | | | | hexanoate |
| 4 | F | (CH₂)₈ | 1-(4-fluorophenyl) | methyl 7- |
| | | | ethan- | (bromotriphenyl- |
| | | | 1-one | phosphaneyl) |
| | | | | heptanoate |
| 5 | F | (CH₂)₉ | 1-(4-fluorophenyl) | methyl 8- |
| | | | ethan-1-one | (bromotriphenyl- |
| | | | | phosphaneyl) |
| | | | | octanoate |
| 6 | Cl | (CH₂)₆ | 1-(4-chlorophenyl) | methyl 5- |
| | | | ethan-1-one | (bromotriphenyl- |
| | | | | phosphaneyl) |
| | | | | pentanoate |
| 7 | Cl | (CH₂)₇ | 1-(4-chlorophenyl) | methyl methyl 6- |
| | | | ethan-1-one | (bromotriphenyl- |
| | | | | phosphaneyl) |
| | | | | hexanoate |
| 8 | Cl | (CH₂)₉ | 1-(4-chlorophenyl) | methyl 8-(bromotriphenyl- |
| | | | ethan-1-one | phosphaneyl)octanoate |
| 9 | Cl | (CH₂)₈ | 1-(4-chlorophenyl) | methyl 7-(bromotriphenyl- |
| | | | ethan-1-one | phosphaneyl)heptanoate |
| 10 | MeO | (CH₂)₉ | 1-(4- | methyl 8-(bromotriphenyl- |
| | | | methoxyphenyl) | phosphaneyl)octanoate |
| | | | ethan-1-one | |
| 11 | Me | (CH₂)₉ | 1-(p-tolyl)ethan- | methyl 8-(bromotriphenyl- |
| | | | 1-one | phosphaneyl)octanoate |

### Example S-02

### Compound 12

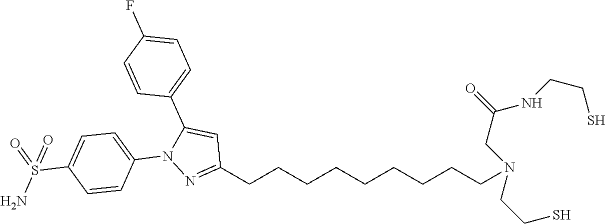 [SMILES: NS(=O)(=O)c1ccc(-n2nc(CCCCCCCCCN(CCS)CC(=O)NCCS)cc2-c2ccc(F)cc2)cc1]

Compound 12 was also prepared by procedures similar to the one described in Example S-01, replacing Intermediate 2 used in Step I with Intermediate 1.

### Example S-03

### Compound 13

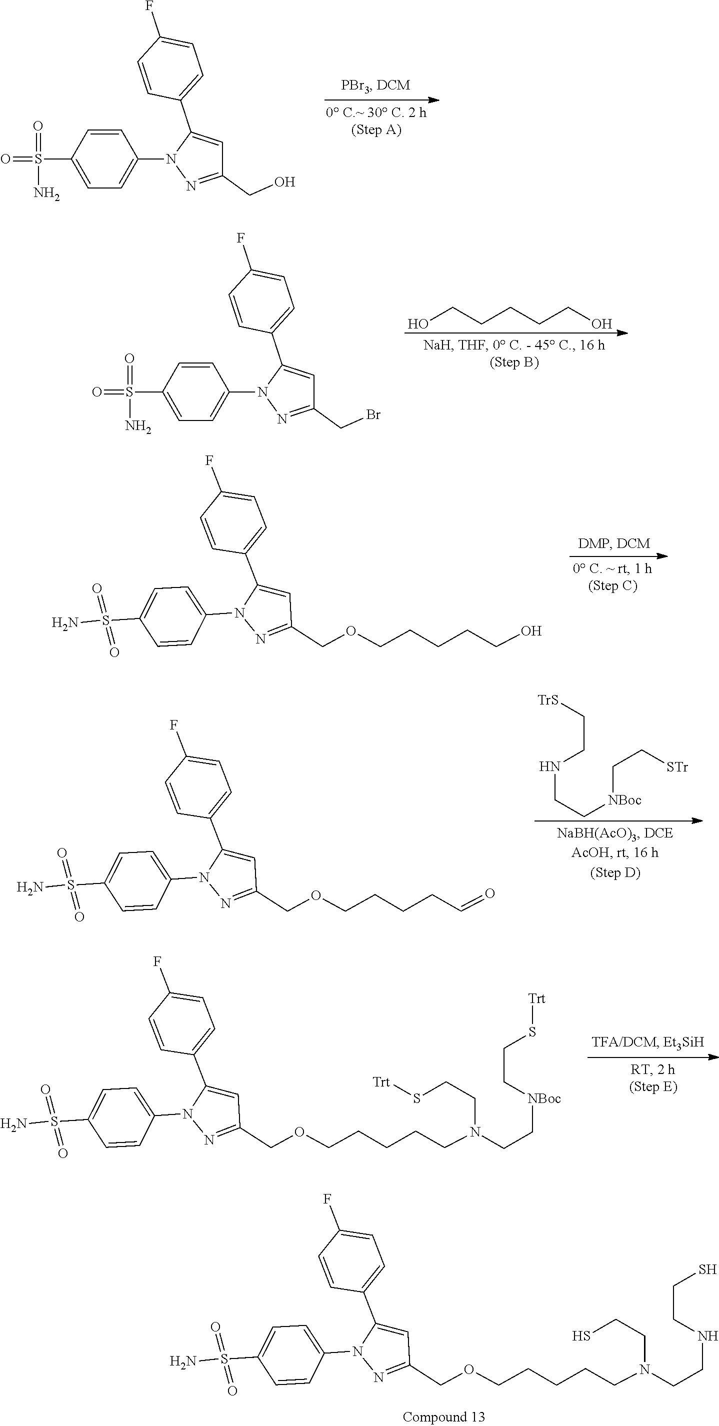

### Step A.

To a solution of 4-(5-(4-fluorophenyl)-3-(hydroxymethyl)-1H-pyrazol-1-yl)benzenesulfonamide (5.5 g, 15.8 mmol) in DCM (250 mL) was added PBr₃ (21.1 g, 79.2 mol) slowly at 0° C. After the reaction was warmed and stirred at 30° C. for 2 h, the reaction was quenched with ice-water (100 ml) and basified with sat. aq. NaHCO₃ solution (100 mL) to adjust the pH to 8. The resulting solution was then extracted with DCM (250 mL×3). The combined organic layers were washed with brine, dried over Na₂SO₄, filtered, and the filtrate was concentrated in vacuo. The residue was purified by silica gel chromatography, eluting with 5-10% MeOH in DCM to give 4-(3-(bromomethyl)-5-(4-fluorophenyl)-1H-pyrazol-1-yl)benzenesulfonamide (4.5 g, yield: 69%) as a yellow solid. Mass Spectrum (ESI) m/z=410 (M+1).

### Step B.

To a solution of pentane-1,5-diol (3.2 g, 30.5 mmol) in dry THE (100 mL) was slowly added NaH (1.22 g, 60% dispersion in mineral oil, 30.5 mmol) at 0° C. After the reaction was stirred at 0° C. for 0.5 h, a solution of 4-(3-(bromomethyl)-5-(4-fluorophenyl)-1H-pyrazol-1-yl) benzenesulfonamide (2.5 g, 6.1 mmol) in THE (20 mL) was added. The resulting mixture was warmed to 45° C. and stirred for 16 h. The reaction was then quenched with sat. aq. NH₄Cl solution (100 ml) and extracted with EtOAc (100 mL×3). The combined organic layers were washed with brine, dried over Na₂SO₄, filtered, and the filtrate was concentrated in vacuo. The residue was purified by silica gel chromatography, eluting with 5-10% MeOH in DCM to give 4-(5-(4-fluorophenyl)-3-(((5-hydroxypentyl)oxy)methyl)-1H-pyrazol-1-yl)benzenesulfonamide (2.0 g, yield: 75%) as a light yellow solid. Mass Spectrum (ESI) m/z=434 (M+1).

### Step C.

To a solution of 4-(5-(4-fluorophenyl)-3-(((5-hydroxypentyl)oxy)methyl)-1H-pyrazol-1-yl)benzenesulfonamide (1.0 g, 2.3 mmol) in DCM (50 mL) was added Dess-Martin periodinane (DMP) (1.95 g, 4.6 mmol) slowly at 0° C. After the reaction was stirred at 0° C. for 1 h, the reaction was quenched by Na₂SO₃ (sat. aq., 25 mL), followed by NaHCO₃ (sat. aq., 25 mL) and extracted with DCM (100 mL×3). The combined organic layers were washed with brine, dried over Na₂SO₄, and concentrated in vacuo to give 4-(5-(4-fluorophenyl)-3-(((5-oxopentyl)oxy)methyl)-1H-pyrazol-1-yl)benzenesulfonamide (650 mg, crude, 60% purity) as a yellow solid, which was used in next step without further purification. Mass Spectrum (ESI) m/z=432 (M+1).

### Step D.

To a solution of 4-(5-(4-fluorophenyl)-3-(((5-oxopentyl)oxy)methyl)-1H-pyrazol-1-yl)benzenesulfonamide (650 mg, crude from last step, ˜0.9 mmol) in DCE (20 mL) was added tert-butyl (2-(tritylthio)ethyl)(2-((2-(tritylthio)ethyl)amino)ethyl)carbamate (532 mg, 0.7 mmol) and 5 drops of CH₃COOH. The resulting mixture was stirred at room temperature for 1 h. Then NaBH(OAc)₃ (1.22 mg, 5.8 mmol) was added and the reaction was stirred at room temperature for another 16 h. Water (50 mL) was added and the reaction was extracted with DCM (50 mL×4). The combined organic layers were washed with brine, dried over Na₂SO₄, and concentrated. The residue was purified by silica gel chromatography, eluting with 10-50% EtOAc in PE to give tert-butyl (2-((5-((5-(4-fluorophenyl)-1-(4-sulfamoylphenyl)-1H-pyrazol-3-yl)methoxy)pentyl)(2-(tritylthio)ethyl)amino)ethyl)(2-(tritylthio)ethyl)carbamate as a white solid (340 mg, yield: 41%). Mass Spectrum (ESI) m/z=1180 (M+1).

### Step E.

To a solution of tert-butyl (2-((5-((5-(4-fluorophenyl)-1-(4-sulfamoylphenyl)-1H-pyrazol-3-yl)methoxy)pentyl)(2-(tritylthio)ethyl)amino)ethyl)(2-(tritylthio)ethyl)carbamate (0.2 g, 0.17 mmol) in DCM/TFA (2:1, 6 mL) was added triethylsilane (20.0 mg, 0.17 mmol) slowly. After the reaction was stirred at room temperature for 2 h, the reaction was concentrated in vacuo to give 4-(5-(4-fluorophenyl)-3-(((5-((2-mercaptoethyl)(2-((2-mercaptoethyl)amino)ethyl)amino)pentyl) oxy)methyl)-1H-pyrazol-1-yl) benzenesulfonamide (100 mg, crude, 70% purity) as a yellow solid, to give Compound 13. Mass Spectrum (ESI) m/z=596 (M+1).

Compounds 14-21 were also prepared by procedures similar to the one described in Example S-03, replacing pentane-1,5-diol in step B with the designated reagent shown in Table 2 below.

TABLE 2

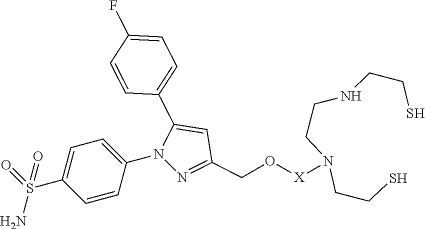

| Compound | R⁴ | X | Reagent used in Step B |
|---|---|---|---|
| 14 | F | (CH₂)₆ | hexane-1,6-diol |
| 15 | F | (CH₂)₄ | butane-1,4-diol |
| 16 | F | (CH₂)₇ | heptane-1,7-diol |
| 17 | F | (CH₂)₃O(CH₂)₃ | 3,3′-oxybis(propan-1-ol) |
| | | | (3,3′-dihydroxydipropyl |
| | | | ether) |
| 18 | Cl | (CH₂)₆ | hexane-1,6-diol |
| 19 | MeO | (CH₂)₆ | hexane-1,6-diol |
| 20 | MeO | (CH₂)₇ | heptane-1,7-diol |
| 21 | Me | (CH₂)₇ | heptane-1,7-diol |

### Example S-04

### Compound 22

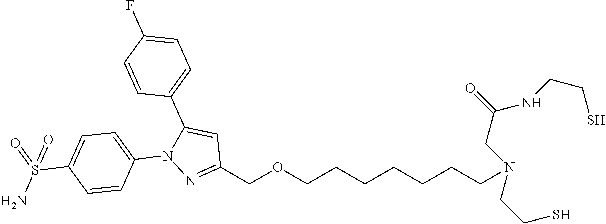 [SMILES: NS(=O)(=O)c1ccc(-n2nc(COCCCCCCCN(CCS)CC(=O)NCCS)cc2-c2ccc(F)cc2)cc1]

Compound 22 was also prepared by procedures similar to the ones described in Example S-03, replacing pentane-1,5-diol in Step B with heptane-1,7-diol and replacing Intermediate 2 used in Step D with Intermediate 1.

### Example S-05

### Compound 23

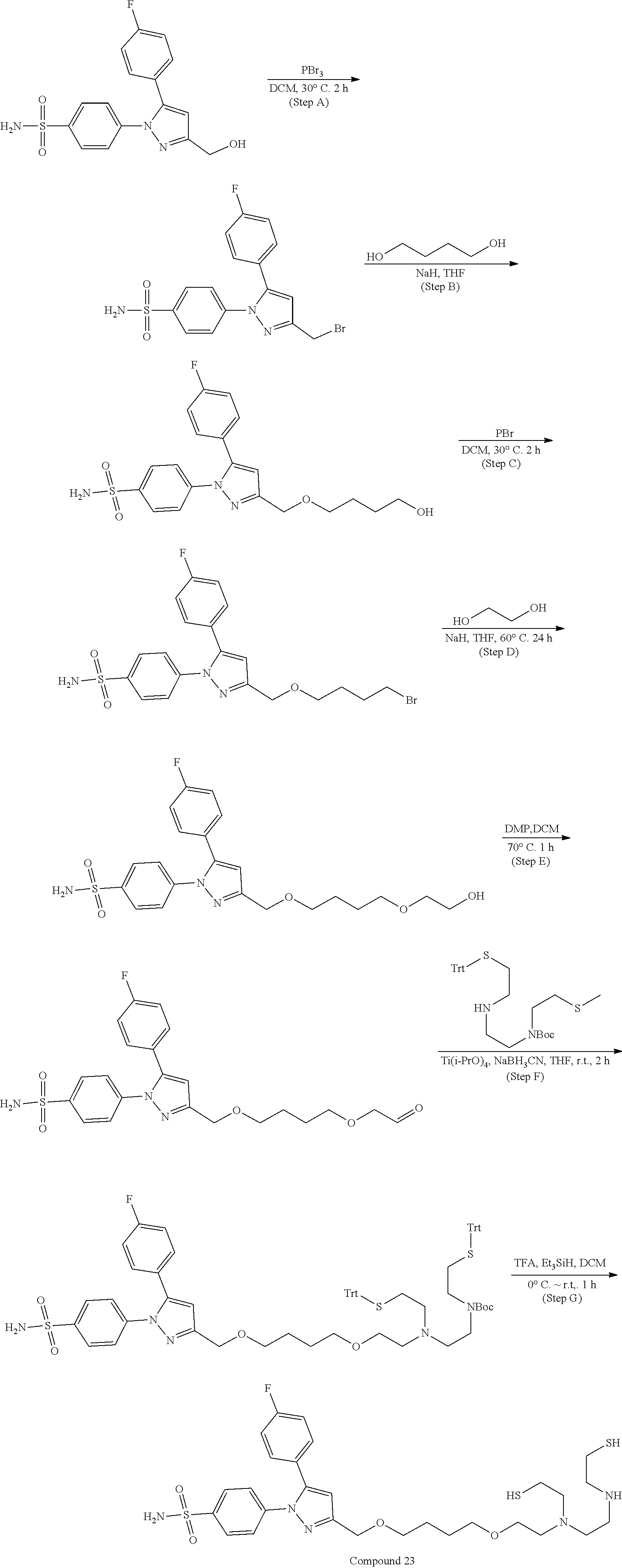

### Step A.

To a solution of 4-(5-(4-fluorophenyl)-3-(hydroxymethyl)-1H-pyrazol-1-yl)benzenesulfonamide (11 g, 32 mmol) in DCM (500 mL) was added PBr₃ (43 g, 160 mmol) at 0° C. After the mixture was warmed and stirred at 30° C. for 2 h. The reaction was quenched with NaHCO₃ (sat. aq., 200 mL) at 0° C. and then extracted with DCM (200 mL×3). The combined organic layers were washed with brine, dried over Na₂SO₄, and concentrated in vacuo. The residue was purified by column chromatography on silica gel, eluting with 5-10% MeOH in DCM to give 4-(3-(bromomethyl)-5-(4-fluorophenyl)-1H-pyrazol-1-yl)benzenesulfonamide (8.5 g, yield: 65%) as a yellow solid. Mass Spectrum (ESI) m/z=410 (M+1).

### Step B.

To a solution of butane-1,4-diol (13 g, 145 mmol) in dry THE (150 mL) was slowly added NaH (5.8 g, 60% dispersion in mineral oil, 145 mmol) at 0° C. After the reaction mixture was stirred at 0° C. for 0.5 h, a solution of 4-(3-(bromomethyl)-5-(4-fluorophenyl)-1H-pyrazol-1-yl)benzenesulfonamide (12 g, 29.2 mmol) in THE (100 mL) was added. The resulting mixture was stirred at room temperature until consumption of starting material as monitored by TLC. On completion, the reaction mixture was quenched by adding sat. aq. NH₄Cl solution (200 mL) at 0° C. The mixture was extracted with EtOAc (200 mL×3). The combined organic layers were washed with brine (200 mL), dried over Na₂SO₄, filtered and the filtrate was concentrated in vacuo. The residue was purified by column chromatography on silica gel, eluting with 0˜70% EtOAc in PE to give 4-(5-(4-fluorophenyl)-3-((4-hydroxybutoxy)methyl)-1H-pyrazol-1-yl)benzenesulfonamide (7 g, yield: 57%) as colorless oil. Mass Spectrum (ESI) m/z=420 (M+1).

### Step C.

To a solution of 4-(5-(4-fluorophenyl)-3-((4-hydroxybutoxy)methyl)-1H-pyrazol-1-yl)benzenesulfonamide (7 g, 16.7 mmol) in DCM (200 mL) was added PBr₃ (22.3 g, 83.5 mmol) slowly at 0° C. The mixture was warmed to 30° C. and stirred at this temperature for 2 h. The reaction mixture was quenched by NaHCO₃ (sat. aq., 100 mL) at 0° C. and then extracted with DCM (200 mL×3). The combined organic layers were washed with brine, dried over Na₂SO₄, and concentrated in vacuo. The residue was purified by column chromatography on silica gel, eluting with 0˜40% EtOAc in PE to give 4-(3-((4-bromobutoxy)methyl)-5-(4-fluorophenyl)-1H-pyrazol-1-yl)benzenesulfonamide (3 g, yield: 38%) as a white solid. Mass Spectrum (ESI) m/z=482 (M+1).

### Step D.

To a solution of ethane-1,2-diol (1.9 g, 31.2 mmol) in THE (20 mL) was added NaH (1.25 g, 31.2 mmol, 60% dispersion in mineral oil) at 0° C. After the mixture was warmed to room temperature and stirred at this temperature for 30 min, a solution of 4-(3-(bromomethyl)-5-(4-fluorophenyl)-1H-pyrazol-1-yl)benzenesulfonamide (3 g, 6.2 mmol) in THE (20 mL) was added. The resulting mixture was warmed to 60° C. and stirred for 24 h. The reaction mixture was then cooled to room temperature, quenched with sat. aq. NH₄Cl solution (10 mL) and then extracted with EtOAc (100 mL×3). The combined organic layers were washed with brine, dried over Na₂SO₄, and concentrated in vacuo. The residue was purified by column chromatography on silica gel, eluting with 0˜70% EtOAc in PE to give 4-(5-(4-fluorophenyl)-3-((4-(2-hydroxyethoxy)butoxy)methyl)-1H-pyrazol-1-yl)benzenesulfonamide (1.4 g, yield: 48%) as colorless oil. Mass Spectrum (ESI) m/z=464 (M+1).

### Step E.

A mixture of 4-(5-(4-fluorophenyl)-3-((4-(2-hydroxyethoxy)butoxy)methyl)-1H-pyrazol-1-yl)benzenesulfonamide (1.4 g, 3.1 mmol) and 2-iodoxybenzoic acid (1.7 g, 6.2 mmol) in MeCN (30 mL) was stirred at 70° C. for 2 h. The mixture was cooled to room temperature, quenched with NaHCO₃ (sat. aq., 30 mL) and NaS₂O₃ (sat. aq., 30 mL) and then extracted with DCM (100 mL×3). The combined organic layers were washed with brine, dried over Na₂SO₄, filtered and the filtrate was concentrated to give 4-(5-(4-fluorophenyl)-3-((4-(2-oxoethoxy)butoxy)methyl)-1H-pyrazol-1-yl) benzenesulfinamide (1.35 g, crude, 50% purity) as a yellow solid. Mass Spectrum (ESI) m/z=462 (M+1).

### Step F.

To a solution of 4-(5-(4-fluorophenyl)-3-((4-(2-oxoethoxy)butoxy)methyl)-1H-pyrazol-1-yl)benzenesulfinamide (1.35 g, crude, ˜1.46 mmol) in THE (30 mL) was added tert-butyl (2-(trityl thio)ethyl)(2-((2-(tritylthio)ethyl)amino)ethyl)carbamate (1.1 g, 1.46 mmol) and Ti(i-PrO)₄ (4.3 g, 15.0 mmol). The resulting solution was stirred at room temperature for 2 h. Then NaBH₃CN (0.6 g, 8.7 mmol) and MeOH (2 mL) were added, and the reaction mixture was stirred for another 10 min. NH₄Cl (sat. aq. 60 mL) was added and the mixture was extracted with DCM (60 mL×3). The combined organic layers were washed with brine (50 mL), dried over Na₂SO₄, filtered and the filtrate was concentrated in vacuo. The residue was purified by column chromatography on silica gel, eluting with 0˜60% EtOAc in PE to give tert-butyl (2-((2-(4-((5-(4-fluorophenyl)-1-(4-sulfamoylphenyl)-1H-pyrazol-3-yl)methoxy)butoxy)ethyl)(2-(tritylthio)ethyl)amino)ethyl)(2-(tritylthio)ethyl) carbamate (220 mg, yield: 6% over 2 steps) as a white solid. Mass Spectrum (ESI) m/z=1210 (M+1).

### Step G.

To a solution of tert-butyl (2-((2-(4-((5-(4-fluorophenyl)-1-(4-sulfamoylphenyl)-1H-pyrazol-3-yl)methoxy)butoxy)ethyl)(2-(tritylthio)ethyl)amino)ethyl)(2-(tritylthio)ethyl) carbamate (100 mg, 0.08 mmol) in DCM (2 mL) was added TFA (1 mL) and Et₃SiH (18 mg, 0.16 mmol) at 0° C. After the mixture was stirred at rt for 1 h, the mixture was concentrated in vacuo to give 4-(5-(4-fluorophenyl)-3-(15-mercapto-10-(2-mercaptoethyl)-2,7-dioxa-10,13-diazapentadecyl)-1H-pyrazol-1-yl)benzenesulfonamide (50 mg, crude, 70% purity) as a pale solid. Mass Spectrum (ESI) m/z=626 (M+1).

### Example S-06

### Compound 24

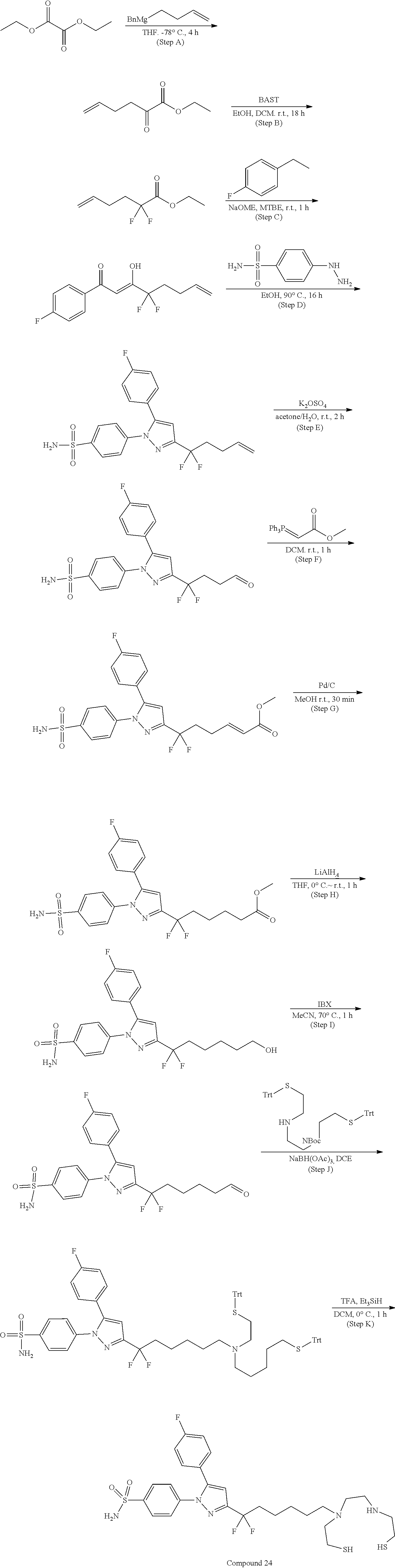

### Step A.

To a solution of diethyl oxalate (5.0 g, 34.2 mmol) in THE (50 mL) was added but-3-en-1-ylmagnesium bromide (82 mL, 0.5 M in THF, 41 mmol) dropwise at −78° C. under N₂. After the reaction was stirred at −78° C. for 4 h, the reaction mixture was quenched with NH₄Cl (sat. aq., 100 mL) at −78° C., then warmed to room temperature and extracted with EtOAc (100 mL×3). The combined organic layers were washed with brine (100 mL), dried over Na₂SO₄, filtered and the filtrate was concentrated in vacuo. The residue was purified by column chromatography on silica gel, eluting with 0˜20% EtOAc in PE to afford ethyl 2-oxohex-5-enoate (2.5 g, yield: 47%) as yellow oil. Mass Spectrum (ESI) m/z=157 (M+1).

### Step B.

To a solution of ethyl 2-oxohex-5-enoate (2.5 g, 16 mmol) in DCM (50 mL) was added bis(2-methoxyethyl)aminosulfur trifluoride (BAST, 6.0 g, 27.2 mmol) at 0° C. Then EtOH (147 mg, 3.2 mmol) was added. The resulting mixture was warmed to room temperature and stirred at this temperature for 16 h. The mixture was quenched by NaHCO₃ (sat. aq., 50 mL) at 0° C. and then extracted with DCM (40 mL×3). The combined organic layers were washed with brine (100 mL), dried over Na₂SO₄, filtered and the filtrate was concentrated under reduced pressure at 0° C. The residue was purified by column chromatography on silica gel, eluting with 0˜20% EtOAc in PE to afford ethyl 2,2-difluorohex-5-enoate (2.0 g, yield: 70%) as yellow oil. No MS.

### Step C.

To a solution of ethyl 2,2-difluorohex-5-enoate (1.9 g, 10.7 mmol) in MTBE (10 mL) was added a solution of 1-(4-fluorophenyl)ethan-1-one (1.3 g, 9.6 mmol) in MTBE (20 mL) and NaOMe (2.05 g, 30% in MeOH, 11.4 mmol). After the resulting mixture was stirred at room temperature for 1 h, the mixture was quenched by HCl (1.0 M aq., 20 mmol) and then extracted with EtOAc (40 mL×3). The combined organic layers were washed with brine (50 mL), dried over Na₂SO₄, filtered and the filtrate was concentrated in vacuo. The residue was purified by column chromatography on silica gel, eluting with 0˜20% EtOAc in PE to afford (Z)-4,4-difluoro-1-(4-fluorophenyl)-3-hydroxyocta-2,7-dien-1-one (1.8 g, yield: 69%) as yellow oil. Mass Spectrum (ESI) m/z=271 (M+1).

### Step D.

To a solution of (Z)-4,4-difluoro-1-(4-fluorophenyl)-3-hydroxyocta-2,7-dien-1-one (1.53 g, 5.7 mmol) in ethanol (40 mL) was added 4-hydrazineylbenzenesulfonamide (1.2 g, 6.2 mmol). After the reaction was stirred at 90° C. for 16 h, the reaction was cooled to room temperature. H₂O (60 mL) was added and extracted with EtOAc (50 mL×3). The combined organic layers were washed with brine (50 mL), dried over Na₂SO₄, filtered and the filtrate was concentrated in vacuo. The residue was purified by column chromatography on silica gel, eluting with 0˜50% EtOAc in PE to afford 4-(3-(1,1-difluoropent-4-en-1-yl)-5-(4-fluorophenyl)-1H-pyrazol-1-yl)benzenesulfonamide (2.33 g, yield: 97%) as a yellow solid.

¹H NMR (400 MHz, DMSO) δ 7.87-7.84 (m, 2H), 7.51-7.48 (m, 4H), 7.38-7.35 (m, 2H), 7.29-7.25 (m, 2H), 6.97 (s, 1H), 5.92-5.85 (m, 1H), 5.14-5.00 (m, 2H), 2.45-2.38 (m, 2H), 2.33-2.26 (m, 2H). Mass Spectrum (ESI) m/z=422 (M+1).

### Step E.

A mixture of 4-(3-(1,1-difluoropent-4-en-1-yl)-5-(4-fluorophenyl)-1H-pyrazol-1-yl)benzenesulfonamide (1.89 g, 4.5 mmol), K₂OsO₄ (56 mg, 0.18 mmol) and NaIO₄ (3.85 g, 18 mmol) in acetone (15 mL) and H₂O (15 mL) was stirred at room temperature for 2 h. The mixture was filtered, and filtrate was extracted with DCM (20 mL×3). The combined organic layers were washed with NaS₂O₃ (sat. aq., 20 mL), dried over Na₂SO₄, filtered and the filtrate was concentrated in vacuo to afford 4-(3-(1,1-difluoropent-4-en-1-yl)-5-(4-fluorophenyl)-1H-pyrazol-1-yl)benzenesulfonamide (1.76 g) as yellow oil. Mass Spectrum (ESI) m/z=424 (M+1).

### Step F.

A mixture of 4-(3-(1,1-difluoro-4-oxobutyl)-5-(4-fluorophenyl)-1H-pyrazol-1-yl)benzenesulfonamide (1.76 g, crude) and Methyl (triphenylphosphoranylidene)acetate (1.68 g, 5 mmol) in DCM (30 mL) was stirred at rt for 1 h, the mixture was concentrated in vacuo and the residue was purified by column chromatography on silica gel, eluting with 0˜70% EtOAc in PE to afford methyl 6,6-difluoro-6-(5-(4-fluorophenyl)-1-(4-sulfamoylphenyl)-1H-pyrazol-3-yl)hex-2-enoate (1.42 g, yield: 66% over 2 steps) as yellow oil. Mass Spectrum (ESI) m/z=480 (M+1).

### Step G.

To a solution of methyl 6,6-difluoro-6-(5-(4-fluorophenyl)-1-(4-sulfamoylphenyl)-1H-pyrazol-3-yl)hex-2-enoate (1.42 g, 2.96 mmol) in MeOH (20 mL) was added Pd/C (0.4 g). After the mixture was stirred at room temperature for 30 min under an atmosphere of hydrogen, it was filtered. The filtrate was concentrated in vacuo to give methyl 6,6-difluoro-6-(5-(4-fluorophenyl)-1-(4-sulfamoylphenyl)-1H-pyrazol-3-yl)hexanoate (1.36 g, crude) as yellow oil. Mass Spectrum (ESI) m/z=482 (M+1).

### Step H.

To a solution of methyl 6,6-difluoro-6-(5-(4-fluorophenyl)-1-(4-sulfamoylphenyl)-1H-pyrazol-3-yl)hexanoate (1.36 g, crude) in THE (30 mL) was added LiAlH₄ (214 mg, 5.65 mmol) at 0° C. The mixture was stirred at room temperature for 1 h. The mixture was quenched by addition of Na₂SO₄×10H₂O (4 g) and filtered. The filter cake was washed with THE (50 mL) and the filtrate was concentrated in vacuo. The residue was purified by column chromatography, eluting with 0˜10% MeOH in DCM to afford 4-(3-(1,1-difluoro-6-hydroxyhexyl)-5-(4-fluorophen yl)-1H-pyrazol-1-yl) benzenesulfonamide (880 mg, yield: 65% over 2 steps) as a yellow solid. Mass Spectrum (ESI) m/z=454 (M+1).

### Step I.

To a solution of 4-(3-(1,1-difluoro-6-hydroxyhexyl)-5-(4-fluorophenyl)-1H-pyrazol-1-yl)benzene sulfonamide (830 mg, 1.83 mmol) in MeCN (30 mL) was added 2-Iodoxybenzoic acid (1.03 g, 3.66 mmol). After the reaction was stirred at 70° C. for 1 h, the mixture was cooled to room temperature. NaHCO₃ (sat. aq., 20 mL) and NaS₂03 (sat. aq., 20 mL) were added and the mixture was stirred for 10 min. The mixture was extracted with DCM (50 mL×3). The combined organic layers were concentrated in vacuo to afford 4-(3-(1,1-difluoro-6-oxohexyl)-5-(4-fluorophenyl)-1H-pyrazol-1-yl)benzenesulfonamide (830 mg, crude, 80% purity) as yellow oil. Mass Spectrum (ESI) m/z=452 (M+1).

### Step J.

To a solution of 4-(3-(1,1-difluoro-6-oxohexyl)-5-(4-fluorophenyl)-1H-pyrazol-1-yl)benzenesulfonamide (830 mg, crude from last step) in DCE (20 mL) was added tert-butyl (2-(tritylthio)ethyl)(2-((2-(tritylthio)ethyl)amino)ethyl)carbamate (1.13 g, 1.47 mmol) and 5 drops of CH₃COOH. The mixture was stirred at room temperature for 1 h. Then NaBH(OAc)₃ (1.95 g, 9.2 mmol) was added to above mixture. The mixture was stirred for another 15 h. The reaction mixture was quenched with water (30 mL) and extracted with DCM (40 mL×4). The combined organic layers were washed with brine, dried over Na₂SO₄, filtered and the filtrate was concentrated in vacuo. The residue was purified by column chromatography on silica gel, eluting with 0˜70% EtOAc in PE to afford tert-butyl (2-((6,6-difluoro-6-(5-(4-fluorophenyl)-1-(4-sulfamoylphenyl)-1H-pyrazol-3-yl)hexyl)(2-(tritylthio)ethyl)amino)ethyl)(2-(tritylthio)ethyl)carbamate (410 mg, yield: 19% over 2 steps) as yellow oil. Mass Spectrum (ESI) m/z=1200 (M+1).

### Step K.

To a solution of tert-butyl (2-((6,6-difluoro-6-(5-(4-fluorophenyl)-1-(4-sulfamoylphenyl)-1H-pyrazol-3-yl)hexyl)(2-(tritylthio)ethyl)amino)ethyl)(2-(tritylthio)ethyl)carbamate (200 mg, 0.17 mmol) in DCM (5 mL)/TFA (3 mL) was added Et₃SiH (40 mg, 0.34 mmol) at 0° C. The mixture was stirred at rt for 1 h. The mixture was concentrated in vacuo to give 4-(3-(1,1-difluoro-6-((2-mercaptoethyl)(2-((2-mercaptoethyl)amino)ethyl)amino)hexyl)-5-(4-fluorophenyl)-1H-pyrazol-1-yl)benzenesulfonamide (100 mg, crude), Compound 24, as a yellow oil. Mass Spectrum (ESI) m/z=616 (M+1).

### Example S-07

### Compound 25

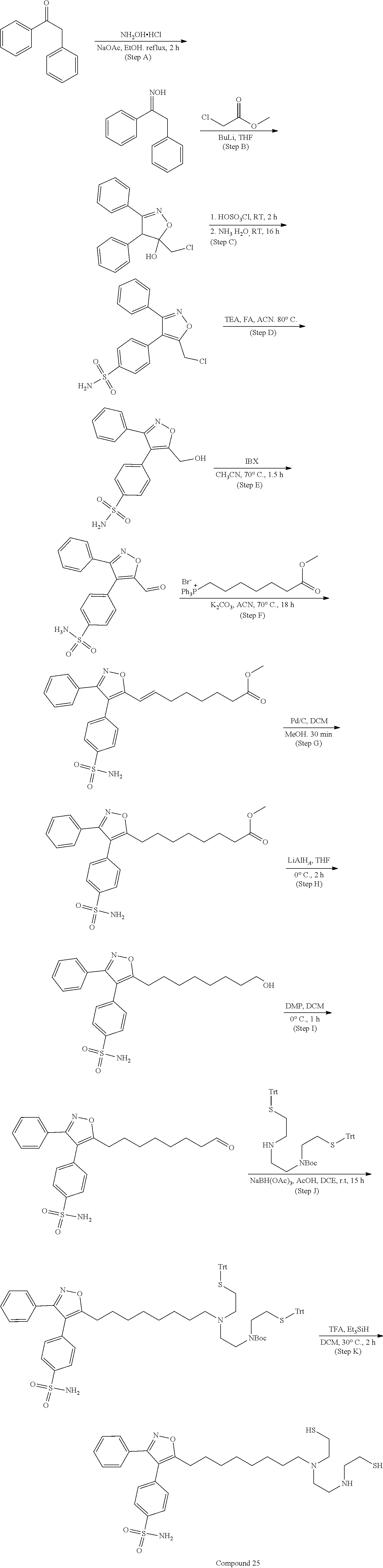

### Step A.

To a solution of deoxybenzoin (1,2-diphenylethan-1-one, 50 g, 250 mmol) in EtOH (250 mL) and H₂O (75 mL) was added hydroxylamine hydrochloride (34.5 g, 500 mmol) and sodium acetate trihydrate (68 g, 500 mmol). The mixture was stirred under reflux for 2 h. The reaction mixture was then diluted with 125 mL of 30% aqueous EtOH and allowed to cool to room temperature whereupon crystals of pure oxime was formed. The product was filtered and dried under reduced pressure to afford 1,2-diphenylethan-1-one oxime as a white solid (50 g, yield: 95%). Mass Spectrum (ESI) m/z=212 (M+1).

### Step B.

To a solution of 1,2-diphenylethan-1-one oxime (10 g, 47.3 mmol) in THE (100 mL) at −78° C. was added butyllithium (42 mL of 2.5M in hexanes, 105 mmol). The internal temperature was maintained below −55° C. during addition. The resulting red solution was warmed to −25° C. and stirred for 1.5 h and then cooled to −78° C. Methyl chloroacetate (11.4 g, 105 mmol) was added. An exotherm was noted and the internal reaction temperature rose to −40° C. The cooling bath was removed, NH₄Cl (sat. aq., 100 mL) and EtOAc (200 mL) were added. The mixture was stirred, and the organic layer was collected. The organic layer was washed again with NH₄Cl (sat. aq., 100 mL) followed by brine (100 mL). The separated organic layer was dried over Na₂SO₄, filtered and the filtrate was concentrated in vacuo. The residue was purified by silica gel chromatography, eluting with PE/EtOAc (93/7) to give 5-(chloromethyl)-3,4-diphenyl-4,5-dihydroisoxazol-5-ol as a light yellow solid (11 g, yield: 81%). Mass Spectrum (ESI) m/z=288 (M+1).

### Step C.

Chlorosulfonic acid (89 g, 764 mmol) was cooled to 0° C. and 5-(chloromethyl)-3,4-diphenyl-4,5-dihydroisoxazol-5-ol (11 g, 38.2 mmol) was added at a rate to keep the internal reaction temperature below 5° C. After the reaction was stirred for 2 h at 20° C., the mixture was poured onto ice and kept the internal reaction temperature below 15° C. EtOAc (200 mL) was added, and the solution was stirred for 15 min. The EtOAc layer was separated and washed with brine (100 mL). NH₄OH (sat, aq., 100 mL) was added to EtOAc layer and resulting mixture was stirred at room temperature for 16 h. The EtOAc layer was separated, dried with Na₂SO₄, filtered and the filtrate was concentrated in vacuo. The residue was purified by silica gel chromatography, eluting with PE/EtOAc (70/30) to give a brown oil. This brown oil was a mixture of meta and para sulfonamides, the pure para-sulfonamide was obtained after two times of recrystallization from isopropyl alcohol (5 g, yield: 37.5%) as a brown solid. ¹H NMR (400 MHz, CDCl₃) δ 7.97 (d, J=8.4 Hz, 2H), 7.46-7.33 (m, 7H), 5.23 (s, 2H), 4.60 (s, 2H). Mass Spectrum (ESI) m/z=349 (M+1).

### Step D.

4-(5-(chloromethyl)-3-phenylisoxazol-4-yl)benzenesulfonamide (5 g, 14.3 mmol), formic acid (2.9 g, 64.3 mmol) and triethylamine (3.6 g, 35.7 mmol) were heated to reflux in acetonitrile (50 mL) for 5 h. The pH of the solution was adjusted to 11 with NaOH (2.5 M aq., −20 mL) and heated to reflux for 3 h then cooled to room temperature. EtOAc (100 mL) and H₂O (80 mL) were added, and the pH of the solution was adjusted to 2 by the addition of concentrated HCl. The layers were separated, and the organic layer was collected, washed with brine (100 mL), dried over Na₂SO₄, filtered and the filtrate was concentrated in vacuo. The residue was purified by silica gel chromatography, eluting with PE/EtOAc (60/40) to give 4-(5-(hydroxymethyl)-3-phenylisoxazol-4-yl)benzenesulfonamide (3.3 g, yield: 69%) as a light yellow solid. ¹H NMR (400 MHz, DMSO) δ 7.88-7.80 (m, 2H), 7.51-7.39 (m, 7H), 7.40-7.34 (m, 2H), 5.78 (t, J=5.8 Hz, 1H), 4.56 (d, J=5.5 Hz, 2H). Mass Spectrum (ESI) m/z=331 (M+1).

### Step E.

To solution of 4-(5-(hydroxymethyl)-3-phenylisoxazol-4-yl)benzenesulfonamide (3.3 g, 10 mmol) in acetonitrile (50 mL) was added 2-iodoxybenzoic acid (IBX) (5.6 g, 20 mmol). The resulting reaction mixture was stirred at 70° C. for 1.5 h then cooled to room temperature. The reaction was quenched with Na₂S₂O₃ (sat. aq., 50 mL), followed by NaHCO₃ (sat. aq., 50 mL) and then extracted with EtOAc (100 mL×3). The combined organic layers were washed with brine, dried over Na₂SO₄, filtered and the filtrate was concentrated in vacuo. The residue was purified by silica gel chromatography, eluting with PE/EtOAc (50:50) to give the 4-(5-formyl-3-phenylisoxazol-4-yl)benzenesulfonamide as a white solid (2.5 g, yield: 76%). Mass Spectrum (ESI) m/z=329 (M+1).

### Step F.

To a solution of 4-(5-formyl-3-phenylisoxazol-4-yl)benzenesulfonamide (2.5 g, 7.6 mmol) in acetonitrile (50 mL) was added methyl 7-(bromotriphenyl-phosphanyl)heptanoate (4.4 g, 9.2 mmol) and K₂CO₃ (2.8 g, 20 mmol) slowly at room temperature. The resulting reaction was stirred at 80° C. for 16 h then cooled to room temperature. H₂O (100 mL) was added, and the mixture was extracted with EtOAc (100 mL×3). The combined organic layers were washed with brine, dried over Na₂SO₄, filtered and the filtrate was concentrated. The residue was purified by silica gel chromatography, eluting with PE/EtOAc (1/1) to give methyl 8-(3-phenyl-4-(4-sulfamoylphenyl)isoxazol-5-yl)oct-7-enoate as a light yellow oil (2.5 g, yield: 72%). Mass Spectrum (ESI) m/z=455 (M+1).

### Step G.

To a solution of methyl 8-(3-phenyl-4-(4-sulfamoylphenyl)isoxazol-5-yl)oct-7-enoate (2.5 g, 5.5 mmol) in MeOH (50 mL) was added Pd/C (200 mg, 10%) at room temperature. After the reaction was stirred under an atmosphere of H₂ at room temperature for 1 h, the reaction was filtered through a plug of Celite® (J. T. Baker, Phillipsberg, NJ, diatomaceous earth) and filter cake was washed with MeOH (50 ml). The filtrate was concentrated in vacuo and purified by silica gel chromatography with PE/EtOAc (1/1) to give methyl 8-(3-phenyl-4-(4-sulfamoylphenyl)isoxazol-5-yl)octanoate as a colorless oil (1.5 g, yield: 60%). Mass Spectrum (ESI) m/z=457 (M+1).

### Step H.

To the solution of methyl 8-(3-phenyl-4-(4-sulfamoylphenyl)isoxazol-5-yl)octanoate (1.5 g, 3.3 mmol) in dry THE (500 mL) was added LiAlH₄ (0.25 g, 6.6 mmol) slowly at 0° C. After the reaction mixture was warmed to room temperature and stirred for 1 h, the reaction was quenched with Na₂SO₄×10H₂O (2 g). The resulting suspension was filtered, the filter cake was washed with THE (50 mL) and EtOAc (50 mL). The combined filtrate was concentrated in vacuo, the residue was purified by silica gel chromatography with MeOH/DCM (1/10) to give 4-(5-(8-hydroxyoctyl)-3-phenylisoxazol-4-yl)benzenesulfonamide as a white solid (1.1 g, yield: 77%). Mass Spectrum (ESI) m/z=429 (M+1).

### Step I.

To a solution of 4-[5-(8-hydroxyoctyl)-3-phenyl-1,2-oxazol-4-yl]benzenesulfonamide (1.1 g, 2.57 mmol) in DCM (80 mL) was added Dess-Martin periodinane (DMP) (2.2 g, 5.14 mmol) slowly at 0° C. After the reaction was stirred at 0° C. for 1 h, the reaction was quenched with Na₂SO₃ (sat. aq., 50 mL), followed by NaHCO₃ (sat. aq., 50 mL) and then extracted with DCM (100 mL×3). The combined organic layers were washed with brine, dried over Na₂SO₄, and concentrated in vacuo to give the crude 4-[5-(8-oxooctyl)-3-phenyl-1,2-oxazol-4-yl]benzenesulfonamide as a yellow solid which was used into next step without further purification (1.2 g, crude, 60% purity), which was used in next step without purification. Mass Spectrum (ESI) m/z=427 (M+1).

### Step J.

To a solution of 4-[5-(8-oxooctyl)-3-phenyl-1,2-oxazol-4-yl]benzenesulfonamide (1.2 g, crude from last step) in DCE (50 mL) was added Intermediate 1 (1.4 g, 1.84 mmol) and 5 drops of CH₃COOH. The resulting solution was stirred room temperature for 1 h. Then NaBH(OAc)₃ (2.4 g, 11.50 mmol) was added and the reaction mixture was stirred for another 16 h. Water (100 mL) was added and the mixture was extracted with DCM (100 mL×3). The combined organic layers were washed with brine, dried over Na₂SO₄, and concentrated. The residue was purified by silica gel chromatography with PE/EtOAc (1/1) to tert-butyl N-[2-({8-[3-phenyl-4-(4-sulfamoylphenyl)-1,2-oxazol-5-yl]octyl}({2-[(triphenylmethyl)sulfanyl]ethyl})amino)ethyl]-N-{2-[(triphenylmethyl)sulfanyl]ethyl}carbamate as a white solid (0.9 g, yield: 30% over 2 steps). Mass Spectrum (ESI) m/z=1175 (M+1).

### Step K.

To a solution of tert-butyl N-[2-({8-[3-phenyl-4-(4-sulfamoylphenyl)-1,2-oxazol-5-yl]octyl}({2-[(triphenylmethyl)sulfanyl]ethyl})amino)ethyl]-N-{2-[(triphenylmethyl)sulfanyl]ethyl}carbamate (0.2 g, 0.17 mmol) in a mixture solvent of DCM/TFA (2:1, 6 mL) was added a solution of triethylsilane (39.53 mg, 0.34 mmol) slowly. The reaction was stirred at room temperature for 2 h. The mixture was concentrated to give 4-(3-phenyl-5-{8-[(2-sulfanylethyl)({2-[(2-sulfanylethyl)amino]ethyl})amino]octyl}-1,2-oxazol-4-yl)benzenesulfonamide (0.1 g, crude with 60% purity), Compound 25, as a yellow solid. Mass Spectrum (ESI) m/z=591 (M+1).

Compounds 26-29 were also prepared by procedures similar to the one described in Example S-07, replacing methyl 7-(bromotriphenyl-phosphaneyl)heptanoate used in Step F and/or Intermediate 1 in Step J with the reagents shown in Table 3 below.

TABLE 3

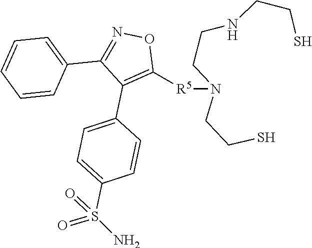

| | | | Reagent used |
|---|---|---|---|
| Example | R | Reagent used in Step F | in Step J |
| 26 | (CH₂)₆ | methyl 5-(bromotriphenyl- | Intermediate 1 |
| | | phosphaneyl)pentanoate | |
| 27 | (CH₂)₇ | methyl 6-(bromotriphenyl- | Intermediate 1 |
| | | phosphaneyl)hexanoate | |
| 28 | (CH₂)₉ | methyl 8-(bromotriphenyl- | Intermediate 1 |
| | | phosphaneyl)octanoate | |
| 29 | (CH₂)₇ | methyl 6-(bromotriphenyl- | Intermediate 2 |
| | | phosphaneyl)hexanoate | |

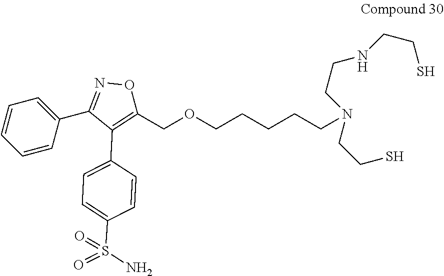 [SMILES: NS(=O)(=O)c1ccc(-c2c(-c3ccccc3)noc2COCCCCCN(CCS)CCNCCS)cc1]

Compound 30 was prepared by procedures similar to the one described in Example S-03, replacing 4-(5-(4-fluorophenyl)-3-(hydroxymethyl)-1H-pyrazol-1-yl)benzenesulfonamide in Step A with 4-(5-(hydroxymethyl)-3-phenylisoxazol-4-yl)benzenesulfonamide (prepared as described in Example S-07, step D) and replacing butane-1,4-diol in Step B with pentane-1,5-diol.

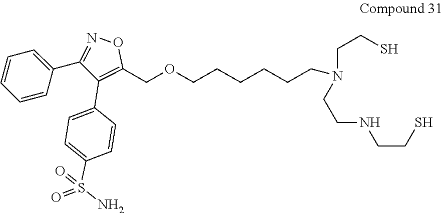 [SMILES: NS(=O)(=O)c1ccc(-c2c(-c3ccccc3)noc2COCCCCCCN(CCS)CCNCCS)cc1]

Compound 31 was prepared by procedures similar to the one described in Example S-03, replacing 4-(5-(4-fluorophenyl)-3-(hydroxymethyl)-1H-pyrazol-1-yl)benzenesulfonamide in Step A with 4-(5-(hydroxymethyl)-3-phenylisoxazol-4-yl)benzenesulfonamide (prepared as described in Example S-07, step D) and replacing butane-1,4-diol in Step B with hexane-1,6-diol.

### Example S-08

### Chelation of Cu

Copper is inserted into the chelators by various methods, such as that described in the following publications: US 2017/0165382; Seo J W et al., “A novel method to label preformed liposomes with ⁶⁴Cu for positron emission tomography (PET) imaging,” Bioconjug Chem. 2008 December; 19(12):2577-84; Taschner I S et al., “Synthesis and structural studies of copper(II) complex with N₂S₂ based N-substituted pendant phosphonic acid arms,” Dalton Trans., 49, 3545-3552, 2020; and Sarkar S et al. “Copper(II) complexes of tetradentate N2S2 donor sets: Synthesis, crystal structure characterization and reactivity,” Polyhedron 28, 1-6, 2009.

### Example S-09

### Chelation of Ga

The complexation of gallium (+3) with bisaminothiols (N2S2 ligands) has been described, for example, in: Inorg. Chem., 35, 6656-6666, (1996); Nuclear Med. Biol., 22, 165-173, (1995); J. Med. Chem., 39, 458-470, (1996); Nucl. Med. Mol. Imaging. 49(3), 208-216, (2015); J. Radioanal. Nucl. Chem., 307, 725-732, (2016).

### Example S-10

### Chelation of Zn and Mn

The complexation of Zn(II) and Mn(II) with chelators has been described, for example, in Inorg. Chimica Acta, 363, 1301-1305, (2010) and J. Chem. Soc. Perkin Trans. 1, 3128-3131).

### BIOLOGICAL EXAMPLES

### Biological Example A

### COX Inhibition Assays

A variety of assays can be used to evaluate inhibition of compounds to cyclooxygenase (COX). Compounds as presently disclosed are screened for inhibition of cyclooxygenase in the following assays.

Cell culture: RAW264.7 murine macrophages are obtained from the Cell Bank of Shanghai Institute of Biochemistry and Cell Biology, Chinese Academy of Sciences (Shanghai, China) and cultured in Dulbecco's modified Eagle's medium containing 100 U/ml penicillin and 100 μg/ml streptomycin at 37° C. in 5% CO₂.

Cell-based COX-2 assay: RAW264.7 cells are plated at a density of 2.5×105/ml cells in a 96-well plate with 0.1 ml of culture medium per well and cultured overnight. The cells are pre-incubated for 30 min with various doses of compounds and stimulated for 7 h with 1 g/ml LPS and 10 U/ml IFN-g. The cell culture supernatants are collected immediately following treatment and centrifuged at 1,000 rpm for 5 min to remove the particulate matter. PGE2 is determined using a Prostaglandin E2 assay kit (catalog no. 62P2APEB; Cisbio Co.). 10 μL of cell supernatant is transferred to a 384-well low volume plate (e. g. Corning® 3544), 5 μL of PGE2-d2 is added, followed by 5 μL anti-PGE2 Cryptate as a negative control. Replace the standard by 10 μL of diluent and PGE2-d2 by 5 μL of reconstitution buffer, Cal0 (for positive control), replace the standard by 10 μL of diluent. Incubate at 4° C. overnight. After centrifuging at 1,000 rpm for 1 min, the dual fluorescence emissions of 615 and 665 nm with a 320 nm excitation are measured using an Envision plate reader (Perkin Elmer, Shelton, CT). The results are expressed as the ratio of 665 nm/615 nm emissions.

COX-1/-2 enzyme assay: The ability of compounds to inhibit ovine COX-1 and human COX-2 is determined using a commercially available enzyme immunoassay (EIA) kit (catalog no. 701090 (COX-1); 701080 (COX-2) Cayman Chemical Co., Ann Arbor, MI, USA) according to the manufacturer's protocol. COX catalyzes the first step in the biosynthesis of AA to PGH2. PGF2α, produced from PGH2 by reduction with stannous chloride, was measured by EIA (ACE™ competitive EIA, Cayman Chemical, Ann Arbor, MI, USA). Briefly, to a series of supplied reaction buffer solutions [960 μl 0.1 M Tris-HCl (pH 8.0) containing 5 mM EDTA and 2 mM phenol] with either COX-1 or COX-2 (10 l) enzyme in the presence of heme (10 μl), 10 μl of various concentrations of test drug solutions are added. These solutions are incubated for 15 min at 37° C. and subsequently 10 μl AA solution (100 μM) is added. The COX reaction is stopped by the addition of 30 μl stannous chloride after 2 min, mixed immediately, supernatants are 2000-fold diluted. The produced PGF2a is measured by EIA. This assay is based on the competition between PGs and a PG-acetylcholinesterase conjugate (PG tracer) for a limited amount of PG antiserum. The amount of PG tracer that is able to bind to the PG antiserum is inversely proportional to the concentration of PGs in the wells since the concentration of the PG tracer is held at a constant while the concentration of PGs varies. The specific antiserum-PG complex binds to a mouse anti-rabbit IgG that had been previously attached to the well. The plate is washed to remove any unbound reagents and 200 μl Ellman's reagent (5,5′-dithiobis-(2-nitrobenzoic acid), which contains the substrate to acetylcholine esterase, is added to the well. The product of this enzymatic reaction generates a distinct yellow color that absorbs at 406 nm. The intensity of this color, determined by spectrophotometry, is proportional to the amount of PG tracer bound to the well, which is inversely proportional to the amount of PGs present in the well during the incubation. Percent inhibition is calculated by the comparison of the compounds treated to the various control incubations.

Dose-response curves are generated using XLFit (IDBS, Surrey, UK) or Prism (GraphPad Software, La Jolla, CA, US) to calculate IC₅₀ values for each compound tested.

### Biological Example B

### “Pain Scans” to Localize Site(s) of Inflammation

A patient with an undiagnosed cause of pain, or a cause of pain which cannot be localized to a site of pathology, is scheduled for a “pain scan.” The patient refrains from drinking or eating for at least eight hours prior to the pain scan. A compound as disclosed herein is administered to the patient either orally or parenterally. After an appropriate period of time determined by the pharmacokinetics of the compound as disclosed herein, during which the compound as disclosed herein binds to cyclooxygenase, the patient is scanned with the appropriate modality to determine the locus or loci of the greatest concentration of the compound. The loci are imaged and viewed or photographed as appropriate. Scans of the patient can be repeated at various intervals after ingestion or injection of the compound as disclosed herein, for example, at two hours, three hours, and four hours after ingestion or injection. The scan findings are correlated with the patient's medical history, physical examination and other information to assist in diagnosis of the etiology of the pain and determine appropriate treatment.

### Biological Example C

### “Tumor Scans” to Localize Site(s) of Tumor(s)

A patient to be screened for presence of a tumor is scheduled for a “COX scan.” The patient refrains from drinking or eating for at least eight hours prior to the COX scan. A compound as disclosed herein is administered to the patient either orally or parenterally. After an appropriate period of time determined by the pharmacokinetics of the compound as disclosed herein, during which the compound as disclosed herein binds to cyclooxygenase, the patient is scanned with the appropriate modality to determine the locus or loci of the greatest concentration of the compound. The loci are imaged and viewed or photographed as appropriate. Scans of the patient can be repeated at various intervals after ingestion or injection of the compound as disclosed herein, for example, at two hours, three hours, and four hours after ingestion or injection. The scan findings are correlated with the patient's medical history, physical examination and other information to assist in diagnosis of the presence and/or location of the tumor and determine appropriate treatment.

### Biological Example D

### Scans to Screen for Asymptomatic Infections or Localized Infections

A patient to be screened for an asymptomatic infection, or to have the site of a localized infection identified, is scheduled for a “COX scan.” The patient refrains from drinking or eating for at least eight hours prior to the COX scan. A compound as disclosed herein is administered to the patient either orally or parenterally. After an appropriate period of time determined by the pharmacokinetics of the compound as disclosed herein, during which the compound as disclosed herein binds to cyclooxygenase, the patient is scanned with the appropriate modality to determine the locus or loci of the greatest concentration of the compound. The loci are imaged and viewed or photographed as appropriate. Scans of the patient can be repeated at various intervals after ingestion or injection of the compound as disclosed herein, for example, at two hours, three hours, and four hours after ingestion or injection. The scan findings are correlated with the patient's medical history, physical examination and other information to assist in diagnosis of the presence and/or location of an infection, and to determine appropriate treatment.

### Biological Example E

### Scans to Screen Candidate Compounds for Imaging

Animal models can be used to test the NSAID derivative compounds disclosed herein for their suitability for clinical use. Animal models of pain (and inflammation related to pain), of infection, and of cancer are well known. See, for example, Handbook of Laboratory Animal Science, Second Edition: Animal Models, Volume 2 (Jann Hau, Gerald L. Van Hoosier Jr., editors), Boca Raton: CRC Press, 2003; Animal Models for the Study of Human Disease (P. Michael Conn, editor), San Diego: Academic Press, 2013.

A suitable animal model (for pain, for cancer, or for infection) is selected and the appropriate pathology is induced. The site of the induced pain, inflammation, infection, or tumor is recorded by the investigator. One or more candidate NSAID derivative compounds disclosed herein is administered to the animal, either by oral gavage or parenterally. After an appropriate period of time determined by the pharmacokinetics of the compound as disclosed herein, during which the compound as disclosed herein binds to cyclooxygenase, the animal is scanned with the appropriate modality to determine the locus or loci of the greatest concentration of the compound. The location(s) indicated by the scan are compared with the known site or sites at which the pathology was induced, for evaluation of the effectiveness of the compound for accumulating at the site of pathology.

The carrageenan induced rat paw edema assay can be used as an exemplary model for inflammation; see Shalini, V. et al., Molecular Immunology 66:229-239 (2015); see also Winter, C. et al., Proc. Soc. Exp. Biol. Med. 111:544-547 (1962). Briefly, acute inflammation is induced by aponeurosis injection of 0.1 ml of 1% carrageenan in 0.9% saline. Additional information regarding model assays is described in Guay et al., J. Biol. Chem. 279:24866-24872 (2004); Nantel et al., British Journal of Pharmacology 128:853-859 (1999); Siebert et al., Proc. Natl. Acad. Sci. USA 91:12013-12017 (1994); de Vries et al., J Nucl. Med. 44:1700-1706 (2003); and Uddin et al., Cancer Prev. Res. 4:1536-1545 (2011).

The animal is then imaged using the appropriate modality, for example, positron emission tomography.

### Biological Example F

### Pharmacokinetic Data

In vitro data of metabolic stability and protein binding as well as in vivo pharmacokinetic data for the NSAID derivative compounds disclosed herein can be generated using the techniques disclosed in Silber, B. M. et al., Pharm. Res. 30(4):932-950 (2013), which is hereby incorporated by reference in its entirety. Various biological, pharmacokinetic and other properties of the NSAID derivative compounds, including hepatic microsomal stability, determination of metabolites, binding to proteins such as plasma protein binding, and in vivo studies, including single-dose and multi-dose pharmacokinetic studies, are determined using the protocols described in that publication and the publications cited therein.

### Biological Example G

### Basophil Activation Testing

The allergenic potential of compounds can be tested using a basophil activation test. The Flow CAST® BAT Assay (Buhlmann Diagnostics Corp, Amherst, New Hampshire, USA, Catalog number FK—CCR—U) can be used for this test. This assay relies on a 2-color flow cytometric detection of activated basophils. In brief, human whole blood is incubated in presence of buffer (background), positive control (IgE or fMLP), or test items (TI). At the same time, cells are stained for activated basophils using the provided staining reagent. In this assay, CCR3 is used as the basophil marker and CD63 as the activation marker. The strategy is to use CCR3 to isolate basophils and then use CD63 to identify activated (CCR3+CD63+) and non-activated (CCR3+CD63−) basophils.

The assay kit provides 2 positive controls, to ensures that donor cells have the capacity to react and provide positive activation signal. This is important as around 15-20% of donors will be negative for one of the controls, and 5-10% negative for both. Donors that do not react to positive stimuli cannot be used to assess allergenic potential of test items. The percentage of activation is determined using the equation:

$$Activated \% = ( \# of CCR 3 + CD 63 + cells ) divided by ( \# of CCR 3 + cells ) \times 100$$

To determine if a compound elicits a positive reaction, result from each donor must be compared to its control conditions. First, non-stimulated donor sample should have less than 5% activated basophils. In addition, one of the two positive controls must give a % activated response above 10%. Finally, the number of analyzed basophils should not be below 200. A % activated response above 10% for a test item is considered a positive response for allergenic potential.

### Biological Example H

### ELISA Histamine Release Assay

This assay is conducted in two parts. First, human whole blood is exposed to the test item, positive control, or negative control to induce the release of histamine from basophils. These conditions are tested for each individual blood sample to compare basal level of histamine and histamine level in test conditions. In addition, an extra treatment group provides the total histamine level for each blood sample (measured after lysis of cells).

Afterward, samples are spun down. Supernatant is collected and acylated for histamine detection. Acylated samples are then submitted to a relatively standard competitive ELISA. Histamine levels in samples incubated with test item and positive controls are then compared to basal and total levels of histamine to evaluate for the presence of an allergenic response.

To evaluate results, donor response to kit controls are first evaluated to ensure the donor is able to produce a valid test result. Spontaneous histamine level must be less than 5% of total release and the positive control must be above 5%. Then to determine if the test item or other compound can induce allergenic response, histamine release levels must be above 5% of total release.

In healthy adults, the reference value for total histamine is ≤60 ng/mL. Thus, if total histamine in a sample is 60 ng/mL, a test item causing a release of greater than 3 ng/mL indicates allergenic potential.

Kits for the ELISA histamine release assay are available from Immuno-Biological Laboratories Inc. (IBL America), Minneapolis, Minnesota, USA. Histamine release kit, catalog number IB89145; Histamine ELISA kit, catalog number IB89128.

### Biological Example I

### Earlier Diagnosis of Rheumatoid Arthritis

Rheumatoid arthritis (RA) is difficult to diagnose especially in the early stages, as the early symptoms are similar to the symptoms of several other diseases and the sensitivity of current methods is inadequate. As a result, at least 30% of patients are not diagnosed at an early stage that could delay or prevent disease progression and severity. It is well established that an early diagnosis of RA with early intervention leads to better patient outcomes. However, currently there are no blood or imaging tests to confirm or rule out an early diagnosis of RA. Diagnosis of RA is about 70% accurate and may not include the extent of the RA throughout the body. Providing a method for accurate early diagnosis of RA will enable treatment to begin earlier in the disease process, can improve patient outcome, and reduce costs associated with the disease.

Imaging with a compound that binds to COX-2, such as the compounds disclosed herein, can significantly improve the sensitivity of the diagnosis and provide guidance on how wide-spread the disease is. A patient usually presents with extremity pain that is non traumatic and with morning stiffness. Because the constellation of joint involvement in RA is not unique in the early stages of the disease, imaging with compounds, such as those disclosed herein, can be used to rule out other causes of autoimmune disorders, leading to more certainty in a diagnosis of RA. For instance, psoriatic arthritis, ankylosing spondylitis, and Reiters syndrome can present only with extremity joint pain. However, it also known that these diseases frequently involve the spine, whereas RA does not. If increased binding of an imaging compound, such as the compounds disclosed herein, is noted in the spinal region on the scan then the diagnosis of RA can be eliminated. In addition, any increased uptake in the kidney could signify inflammation of the kidney that is caused by systemic lupus erythematous (SLE nephritis) which, again, eliminates the diagnosis of RA.

If a clinician suspects a person may have RA, the patient is scheduled for a scan with a compound as disclosed herein. The patient refrains from drinking or eating for at least eight hours prior to the scan. A compound as disclosed herein is administered to the patient either orally or parenterally. After an appropriate period of time determined by the pharmacokinetics of the compound as disclosed herein, during which the compound as disclosed herein binds to cyclooxygenase-2 (COX-2), the patient is scanned with the appropriate modality (e.g., PET) to determine the locus or loci of the greatest concentration of the compound. The loci are imaged and viewed or photographed as appropriate with emphasis on regions typically affected by RA, such as joints in the fingers, and on regions that are involved in other disease processes with early symptoms that mimic RA. Scans of the patient can be repeated at various intervals after ingestion or injection of the compound as disclosed herein, for example, at two hours, three hours, and four hours after ingestion or injection. The scan findings are correlated with the patient's medical history, physical examination and other information to assist in diagnosis.

Therapeutic agents, such as non-steroidal anti-inflammatory agents, steroids, methotrexate, or biologics such as Humira® or Remicade® can be prescribed for patients with overexpression of COX-2 in regions affected by rheumatoid arthritis, including the synovium of various joints.

### Biological Example J

### Evaluating Efficacy of Treatment of Rheumatoid Arthritis

Patients can be treated for rheumatoid arthritis (RA) using several therapies, including various pharmaceutical agents, physical therapy, or surgery. In the United States, approximately 900,000 RA patients per year are treated with anti-TNF antibodies such as Humira®. These treatments are expensive and carry the risk of side effects such as infection. In addition, approximately 40% of patients treated with anti-TNF antibodies stop responding to the treatment within a year. Early determination of the efficacy and of patient response to treatment can thus avoid both side effects and unnecessary costs of treatment.

Imaging agents for COX-2 enzyme levels, such as the compounds disclosed herein, can be used as a companion diagnostic to identify when antibody treatment has stopped working. Imaging scans with such agents can be used on a regular schedule. If the practitioner sees that the COX-2 enzyme levels are not going down, they can discontinue treatment. This would save expense and reduce the patient side effects of the treatment that is no longer working.

A patient undergoing treatment for RA, such as a patient being treated with anti-TNF antibodies, is scheduled for a scan with a compound as disclosed herein. The patient refrains from drinking or eating for at least eight hours prior to the scan. A compound as disclosed herein is administered to the patient either orally or parenterally. After an appropriate period of time determined by the pharmacokinetics of the compound as disclosed herein, during which the compound as disclosed herein binds to cyclooxygenase, the patient is scanned with the appropriate modality to determine the locus or loci of the greatest concentration of the compound. The loci are imaged and viewed or photographed as appropriate with emphasis on regions typically affected by RA, such as joints in the fingers. Scans of the patient can be repeated at various intervals after ingestion or injection of the compound as disclosed herein, for example, at two hours, three hours, and four hours after ingestion or injection. The scan findings are correlated with the patient's medical history, physical examination and other information to assist in diagnosis. Inflammation and overexpression of COX-2 in regions affected by rheumatoid arthritis, such as the synovium of various joints, is determined. The efficacy of treatment is assessed based on these determinations, and the specific treatment can be continued, terminated, or adjusted as appropriate.

### Biological Example K

### Evaluating Need for Opiate Treatment

Physicians currently do not have an objective quantifiable diagnostic tool to determine if a patient actually has pain that requires opioid treatment. Though states have developed guidelines or suggestions on the proper length of time to utilize opioid therapy, it has not been shown that these guidelines are adequate to reliably guide clinical practice. Imaging with an agent that indicates levels of COX-2 enzyme, such as the compounds disclosed herein, represents a more objective method for determining the necessity of opioids.

Opioid misuse is a severe problem in the United State and in other countries, underscoring the importance of ensuring that patients with severe pain are appropriately treated, and also that patients that do not need opioid drugs to control pain are appropriately excluded from opioid treatment. In the United States, over 190 million opioid prescriptions are written per year. The United States is in the midst of an opioid crisis which began because of the significant misuse of prescription opioids. Four out of five heroin users began using heroin after using prescription opioids, underscoring the need for determining when opioid drugs are truly needed.

Pain physicians and primary care doctors do not have an objective and quantifiable way of deciding on writing a prescription for opioids. Imaging with an agent that indicates levels of COX-2 enzyme, such as the compounds disclosed herein, can provide important information on the levels of COX-2 enzyme in the body. If elevated COX-2 is not seen on exam, then an opioid prescription is not indicated. Imaging with agents such as those disclosed herein can play a significant role in reducing the number of prescriptions, while making sure that the patients that truly need opioids are appropriately taken care of.

If a patient presents to a physician with a complaint of pain, and the physician cannot determine the cause of the pain, a “pain scan” as in Biological Example A can be performed. The scan is performed either on the specific location of pain indicated by the patient, or over the entire body of the patient. The amount and distribution of COX-2 expression is determined, enabling the physician to decide whether an opioid prescription or a different treatment is indicated.

The initial scan can serve as a baseline for COX-2 expression for comparison with later scans during future physician visits, to determine if COX-2 expression has remained stable or has changed. If the patient has received a previous prescription for opioid drugs, a comparison of the initial baseline scan with later scans is helpful in determining whether continued treatment with opioid drugs is warranted.

The disclosures of all publications, patents, patent applications and published patent applications referred to herein by an identifying citation are hereby incorporated herein by reference in their entirety.

The present disclosure has been described in terms of specific embodiments incorporating details to facilitate the understanding of principles of construction and operation of the disclosure. Such reference herein to specific embodiments and details thereof is not intended to limit the scope of the claims appended hereto. It will be readily apparent to one skilled in the art that other various modifications can be made in the embodiments chosen for illustration without departing from the spirit and scope of the disclosure. Therefore, the description and examples should not be construed as limiting the scope of the invention.

1. A conjugate compound of Formula (II) or Formula (I): 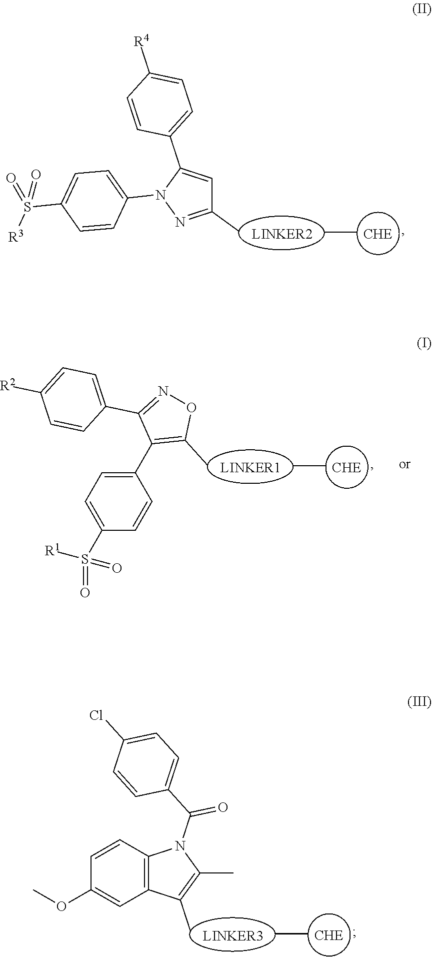
or a salt thereof, wherein:
R¹ is —NH₂ or —CH₃;
R² is H, F, Cl, —CH₃, —OCH₃, or —CF₃;
R³ is —NH₂ or —CH₃;
R⁴ is H, F, Cl, —CH₃, —OCH₃, or —CF₃; 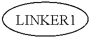
is —R⁵—;
R⁵ is alkylene, haloalkylene, alkenylene, heteroalkylene, or heteroalkylene substituted with halogen; 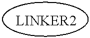
is —R⁶—;
R⁶ is alkylene, haloalkylene, alkenylene, heteroalkylene, or heteroalkylene substituted with halogen; 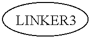
is —R⁷—;
R⁷ is alkylene, haloalkylene, alkenylene, heteroalkylene, heteroalkylene substituted with halogen, —CH₂—C(═O)NH-alkylene, —CH₂—C(═O)NH-haloalkylene, —CH₂—C(═O)NH-alkenylene, —CH₂—C(═O)NH-heteroalkylene, —CH₂—C(═O)NH-heteroalkylene substituted with halogen, —CH₂—NHC(═O)-alkylene, —CH₂—NHC(═O)-haloalkylene, —CH₂—NHC(═O)-alkenylene, —CH₂—NHC(═O)-heteroalkylene, or —CH₂—NHC(═O)-heteroalkylene substituted with halogen; 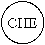
is 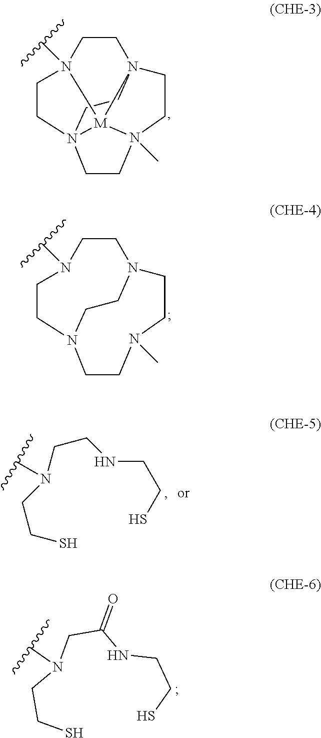
and
M is copper, gallium, zinc, or manganese.

2. The compound of claim 1, wherein the compound is of Formula (II): 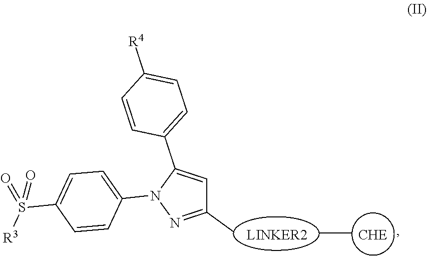
or a salt thereof.

3. The compound of claim 1, wherein the compound is of Formula (I): 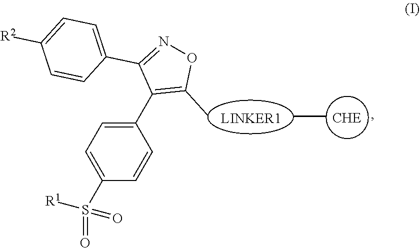
or a salt thereof.

4-47. (canceled)

48. A pharmaceutical composition comprising one or more compounds of claim 1, or a salt thereof, and a pharmaceutically acceptable excipient.

49. A kit comprising one or more compounds selected from of claim 1, or a salt thereof, and printed or electronic instructions for adding a radioactive agent to said compound.

50. A method of imaging a site of pathology or suspected pathology in a subject, comprising:
a) administering one or more compounds of claim 1, or a salt thereof, or the composition of claim 48, to the subject, wherein M is ⁶⁴Cu; and
b) generating an image of the subject or an image of a portion of the subject.

51. The method of claim 50, wherein the pathology or suspected pathology in the subject is a tumor or a suspected tumor.

52. The method of claim 50, wherein the subject is suffering from pain.

53. The method of claim 50, wherein the pathology or suspected pathology in the subject is an infection or a suspected infection.

54-61. (canceled)

62. A method of diagnosing rheumatoid arthritis in a subject, comprising:
a) administering one or more compounds of claim 1 to the subject; and
b) generating an image of at least one synovial joint of the subject
wherein elevated COX-2 expression is indicative of the subject having rheumatoid arthritis.

63. (canceled)

64. A method of determining the efficacy of treatment of rheumatoid arthritis in a subject undergoing treatment for rheumatoid arthritis, comprising:
a) administering one or more COX-2 binding detectable compounds to the subject; and
b) generating an image of at least one synovial joint of the subject wherein a normal COX-2 expression level in the synovial joint is indicative of the efficacy of the treatment;
or
A) prior to commencement of treatment of rheumatoid arthritis in the subject,
i) administering one or more COX-2 binding detectable compounds to the subject;
ii) generating an image of at least one synovial joint of the subject;
B) providing treatment for rheumatoid arthritis to the subject;
C) subsequent to the providing treatment,
i′) administering one or more COX-2 binding detectable compounds to the subject;
ii′) generating an image of at least one synovial joint of the subject; and
D) comparing the image of the synovial joint from step a-ii to the image of the synovial joint from step c-ii′,
wherein a reduction in the level of COX-2 binding compound detected in the synovial joint in the image of the synovial joint from step C-ii′ compared to the level of COX-2 binding compound detected in the image of the synovial joint from step A-ii is indicative of the efficacy of the treatment.

65. (canceled)

66. A method of treating rheumatoid arthritis in a subject, comprising:
a) administering one or more of claim 1 to the subject;
b) generating an image of at least one synovial joint of the subject;
c) determining if COX-2 expression is elevated in the synovial joint; and
d) administering a therapy for rheumatoid arthritis to the subject if COX-2 expression is elevated.

67-68. (canceled)

69. The method of claim 66, wherein the synovial joint displays a symptom associated with rheumatoid arthritis.

70. The method of claim 69, wherein the symptom associated with rheumatoid arthritis is pain, stiffness, swelling, redness, reduced range of motion, sensation of heat, or sensation of burning.

71. The method of claim 69, wherein determining if COX-2 expression is elevated in the synovial joint comprises:
generating an image of a further synovial joint of the subject that is not affected by the symptom of rheumatoid arthritis, where the image of a further synovial joint of the subject is generated prior to, simultaneously with, or subsequent to generating an image of the at least one synovial joint of the subject that displays a symptom associated with rheumatoid arthritis; and
comparing the image of the at least one synovial joint of the subject to the image of the further synovial joint of the subject that is not affected by the symptom of rheumatoid arthritis.

72. The method of claim 66, wherein M is ⁶⁴Cu.

73. A method of determining whether a subject is suffering from pain, comprising:
a) administering one or more of claim 1 to the subject; and
b) generating an image of the subject or an image of a portion of the subject, wherein elevated COX-2 expression is indicative of the subject suffering from pain; or
A) administering one or more COX-2 binding detectable compounds to the subject;
B) generating an image of the subject or an image of a portion of the subject;
C) determining if COX-2 expression is elevated in the subject or the portion of the subject; and
D) administering a therapeutic drug to treat pain in the subject,
wherein elevated COX-2 expression is indicative of the subject suffering from pain.

74-122. (canceled)

123. A compound of claim 1 of the formula: 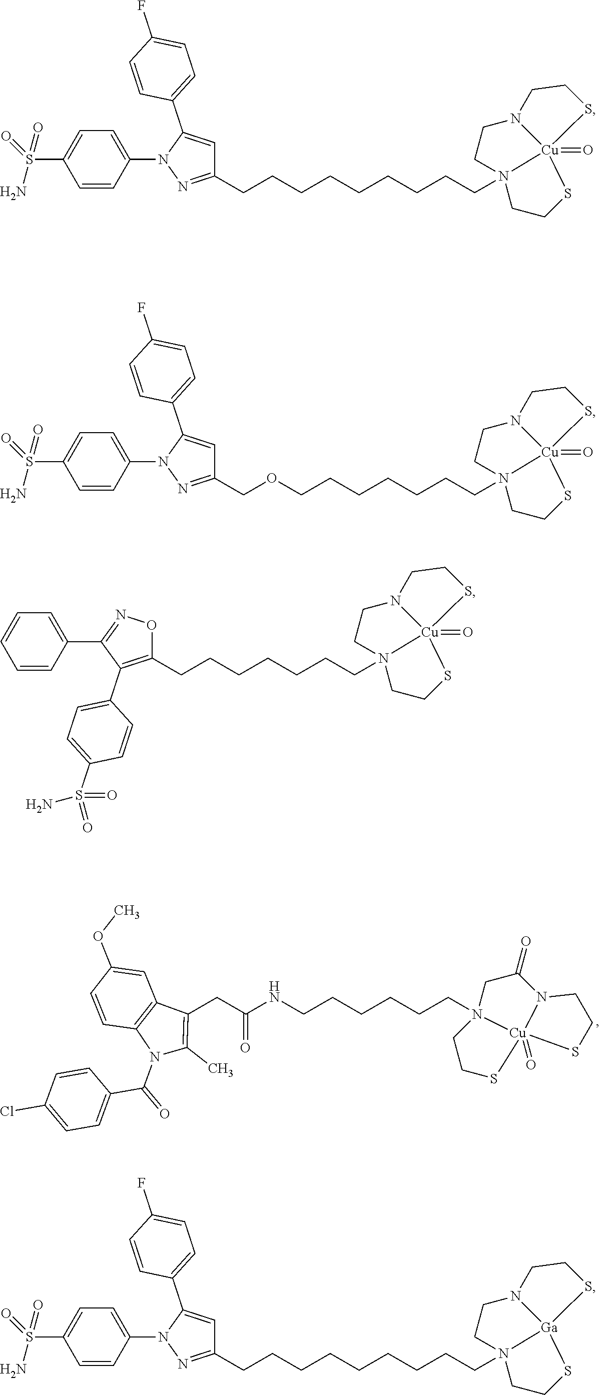 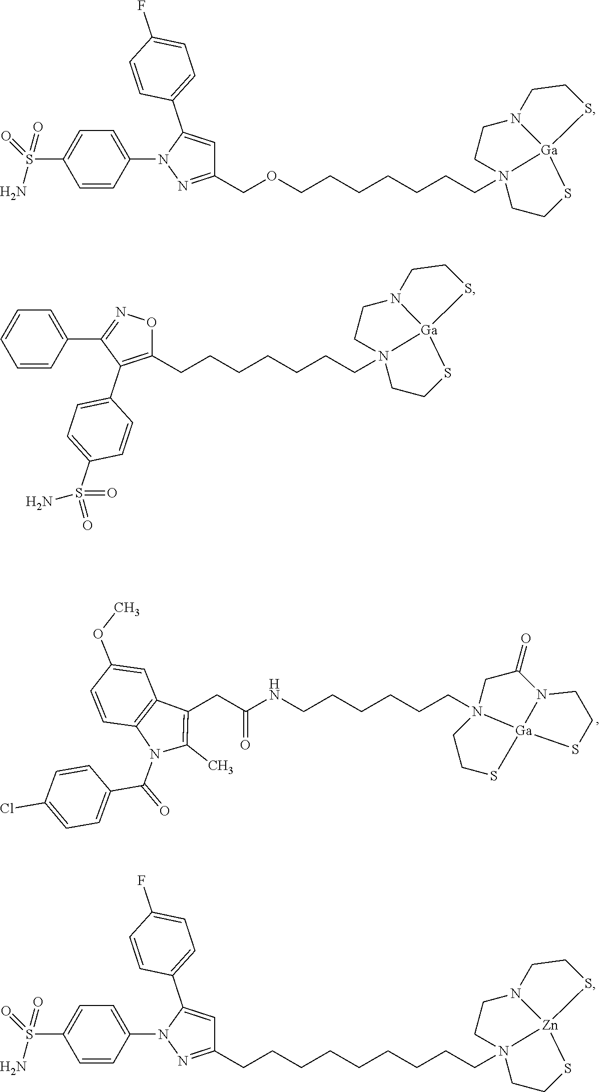 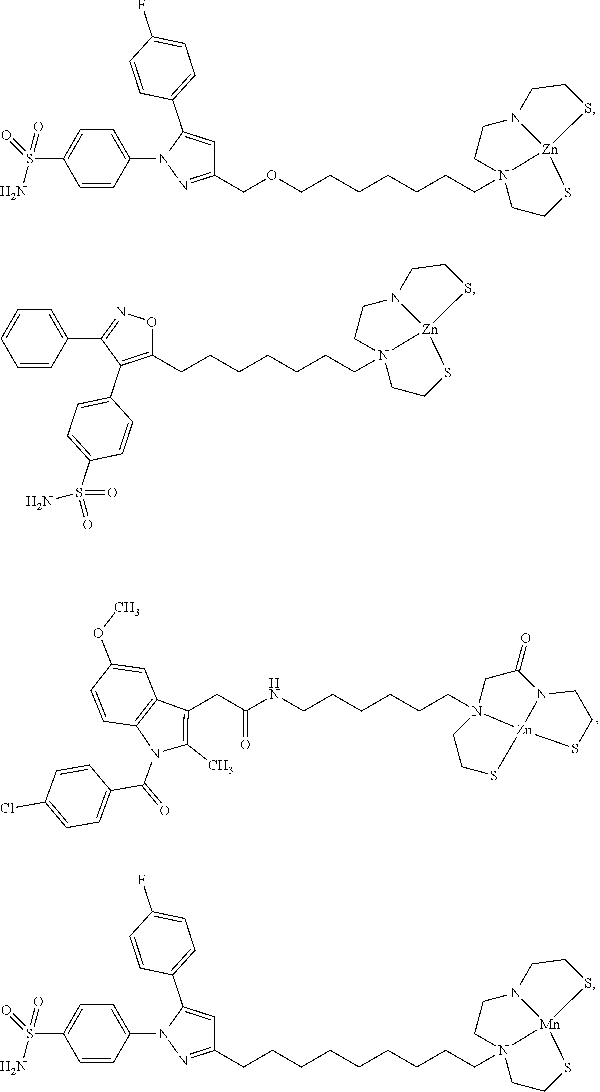 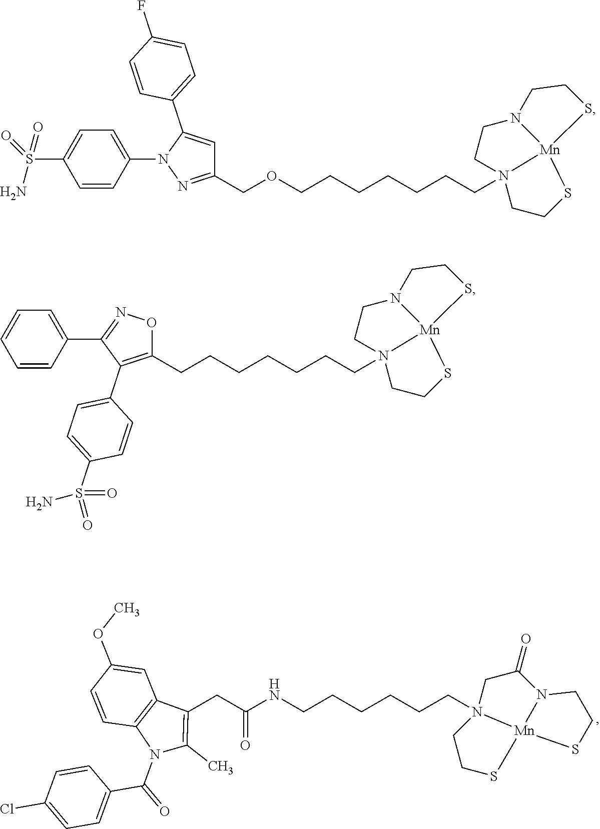
or a salt thereof.

124. A compound of the formula: 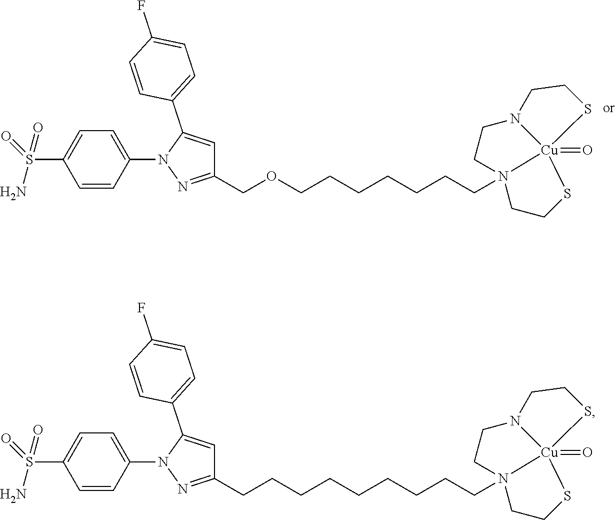
or a salt thereof.

125. A compound of the formula: 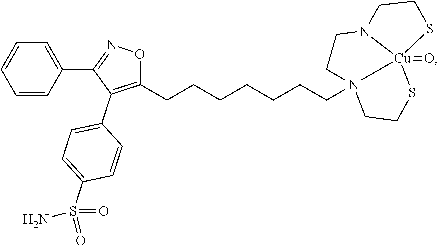
or a salt thereof.

126-188. (canceled)
